(19) United States
(12) Patent Application Publication
(10) Pub. No.: US 2025/0025610 A1
(43) Pub. Date: Jan. 23, 2025

(54) SYSTEM AND METHOD FOR ALLOGENEIC OR XENOGENEIC USE OF ALPHA 2M MOLECULES IN TREATING MEDICAL CONDITIONS

(71) Applicant: Astaria Global, LLC, Houston, TX (US)

(72) Inventors: Daniel J. Dreyfuss, Hampstead, MD (US); William J. Cramer, Houston, TX (US); Michael D. Duvall, Houston, TX (US); Robert S. Shoemaker, Eagle, ID (US)

(21) Appl. No.: 18/909,762

(22) Filed: Oct. 8, 2024

Related U.S. Application Data
(63) Continuation-in-part of application No. 18/909,731, filed on Oct. 8, 2024.
Application No. 18/909,731 is a continuation-in-part of application No. 18/424,552, filed on Jan. 26, 2024.
Application No. 18/424,552 is a continuation-in-part of application No. 18/535,841, filed on Dec. 11, 2023.
Application No. 18/535,841 is a continuation-in-part of application No. 18/215,498, filed on Jun. 28, 2023.
Application No. 18/215,498 is a continuation of application No. 17/837,090, filed on Jun. 10, 2022, now Pat. No. 11,833,279.
(60) Provisional application No. 63/209,685, filed on Jun. 11, 2021.
(60) Provisional application No. 63/467,497, filed on May 18, 2023.
(60) Provisional application No. 63/541,116, filed on Sep. 28, 2023.
(60) Provisional application No. 63/467,497, filed on May 18, 2023.
(60) Provisional application No. 63/541,116, filed on Sep. 28, 2023.

(51) Int. Cl. A61M 1/02; A61J 1/20; A61K 35/16; A61K 38/17; A61K 38/57; B01L 3/00

(52) U.S. Cl. CPC A61M 1/029; A61J 1/201; A61J 1/2062; A61J 1/2082; A61J 1/2096; A61K 35/16; A61K 38/1722; A61K 38/57; B01L 3/5021; B01L 3/50825; A61M 2202/0417; B01L 2300/0832; B01L 2400/0409; B01L 2400/0478

(57) ABSTRACT

A method for treating a medical condition of a patient with at least Alpha-2 Macroglobulin (α2M) molecules in an allogeneic or xenogeneic manner includes: drawing whole blood from a donor; separating platelet poor plasma (PPP) containing α2M molecules from other components of the whole blood; filtering waste plasma from the PPP to generate an aggregate of the α2M molecules; and administering at least some of the aggregate to the patient via injection or inhalation, wherein the donor and the patient are different individuals.

### RELATED APPLICATIONS

This application is a continuation-in-part of U.S. patent application Ser. No. 18/909,731 entitled “SYSTEM AND METHOD FOR AGGREGATION AND AUTOLOGOUS USE OF α2M MOLECULES TO TREAT RESPIRATORY CONDITIONS” filed Oct. 8, 2024 by Daniel J. Dreyfuss et al. (Attorney Docket No. JL1235.021); which is a continuation-in-part of U.S. patent application Ser. No. 18/424,552 entitled “SYSTEM AND METHOD FOR ALLOGENEIC OR XENOGENEIC USE OF ALPHA 2M MOLECULES IN TREATING MEDICAL CONDITIONS” filed Jan. 26, 2024 by Daniel J. Dreyfuss et al. (Attorney Docket No. JL1235.016); which is a continuation-in-part of U.S. patent application Ser. No. 18/535,841 entitled “SYSTEM AND METHOD FOR ISOLATION AND AUTOLOGOUS USE OF ALPHA 2M MOLECULES TO TREAT RESPIRATORY CONDITIONS” filed Dec. 11, 2023 by Daniel J. Dreyfuss et al. (Attorney Docket No. JL1235.015); the disclosures of each of which are incorporated by reference for all purposes.

U.S. patent application Ser. No. 18/909,731 is also a continuation in part of U.S. patent application Ser. No. 18/535,841; which is a continuation-in-part of U.S. patent application Ser. No. 18/215,498 entitled “SYSTEM AND METHOD FOR ISOLATING ALPHA 2M MOLECULES” filed Jun. 28, 2023 by David J. Lutz et al. (Attorney Docket No. JL1235.017); which is a continuation of U.S. patent application Ser. No. 17/837,090 entitled “SYSTEM AND METHOD FOR ISOLATING ALPHA 2M MOLECULES” filed Jun. 10, 2022 by David J. Lutz et al. (Attorney Docket No. JL1235.005, since issued as U.S. Pat. No. 11,833,279); which claims the benefit of the priority date of U.S. Provisional Application 63/209,685 entitled “SYSTEM AND METHOD FOR ISOLATING α2M MOLECULES” filed Jun. 11, 2021 by David J. Lutz et al. (Attorney Docket No. JL1234.004); the disclosures of each of which are incorporated herein by reference for all purposes.

U.S. patent application Ser. Nos. 18/535,841 and 18/424,552 both also claim the benefit of the priority date of each of U.S. Provisional Application 63/467,497 entitled “SYSTEM AND METHOD FOR ISOLATING AND USE OF ALPHA 2M MOLECULES IN TREATING RESPIRATORY CONDITIONS” filed May 18, 2023 by Daniel J. Dreyfuss et al. (Attorney Docket No. JL1235.013); and of U.S. Provisional Application 63/541,116 entitled “SYSTEM AND METHOD FOR ISOLATION AND ALLOGENEIC USE OF α2M MOLECULES TO TREAT MEDICAL CONDITIONS” filed Sep. 28, 2023 by Daniel J. Dreyfuss et al. (Attorney Docket No. JL1235.014); the disclosures of each of which are also incorporated herein by reference for all purposes.

### BACKGROUND

### 1. Technical Field

The present disclosure relates to medical procedures for the aggregation and delivery of Alpha-2 Macroglobulin (α2M) molecules in an allogeneic or xenogeneic manner to treat musculoskeletal and/or respiratory conditions.

### 2. Description of the Related Art

Medical study and research continues to reveal ever more detail about processes that occur in the bodies of human beings and other creatures, including details about the healing process that is triggered in response to injuries to a variety of tissues. By way of example, it has been found that injuries to at least musculoskeletal and respiratory tissues, whether caused by physical trauma, allergy response or disease, triggers a complex combination of both regenerative and destructive activities.

More specifically, there is a combination of growth of new tissue to repair and/or replace damaged tissue, and a remodeling of both old and new tissues to recreate the structure that was damaged with a correct shape and size. The remodeling process is effected by the provision of proteases at the location of the tissue damage to effectively “sculpt” the tissue structures that result from the growth process through the selective breakdown of the proteins making up portions of both old and new tissues. In effect, the healing process is meant to be a balance of both a growth process, and a selective destruction process in which excess portions of tissue are “trimmed” away.

Unfortunately, it is not uncommon for such “sculpting” to go too far as a result of an overabundance and/or hyperactivity of the proteases. The result may be misshapen tissue structures, the excessive formation of fibrotic tissue (e.g., scar tissue) in place of normal tissue, and/or the destruction of existing tissue that is not accompanied by replacement thereof.

In particular regarding musculoskeletal conditions, the result may be misshapen musculoskeletal structures (e.g., misshapen portions of bones), the excessive formation of fibrotic tissue (e.g., scar tissue) in place of normal musculoskeletal structures, and/or the destruction of existing musculoskeletal structures without repair (e.g., partial or complete loss of cartilage at a joint). By way of example, it may be that a physical injury to a joint triggers a misperformed healing process in which the proteases become overactive, thereby damaging the cartilage at the joint, and thereby triggering the onset of arthritis at the joint. The reduction in the cushioning provided by the cartilage may lead to inflammation at the joint, which can repeatedly re-trigger the same misperformed healing process. Over time, this may cause the destruction and loss of all cartilage at the joint, and a misshapening of portions of the bones at the joint.

In particular regarding respiratory conditions, the result may be misshapen air sac structures (e.g., enlarged and floppy air sac structures that are less effective), the excessive formation of fibrotic tissue (e.g., thickened air sac tissue), and/or the destruction of air sac tissue without repair (e.g., a reduction in the overall quantity of air sacs). By way of example, it may be that the inhalation of airborne allergens (e.g., fungi in a dusty feeding trough for horses), and/or particles having a microscopically sharp geometry that causes irritation (e.g., asbestos particles) triggers the inflammation of lung tissue, leading to a misperformed healing process in which the proteases become overactive. This may lead to thickening of air sac tissue that reduces the efficiency of blood oxygenation, and/or that causes the lungs to become less elastic such that physical act of breathing in and out requires more physical exertion.

Further, regarding respiratory conditions, for reasons that have remained unclear, rates of occurrence of various respiratory conditions, including asthma and obstructive pulmonary disease, have been observed to be increasing in recent years for a wide variety of creatures, including in various pets and farm animals (e.g., camels, dogs, horses, pigs, etc.). By way of example, in horses, rates of equine asthma (EA), including recurrent airway obstruction (RAO) and summer pasture-associated obstructive pulmonary disease (SPAOPD), have been observed to be increasing over the last couple of decades. These increasing rates also seem to mirror similar increasing rates of respiratory conditions in humans over a similar period of time.

Where humans are concerned, the increasing rates of respiratory conditions have already prompted increased efforts to discern the causes, and to develop treatments and/or cures. This has already resulted in an increasing variety of treatments available both over the counter and by prescription. A number of these treatments involve the delivery of a saline solution, and/or a solution of a pharmaceutical, in aerosolized form (e.g., through the use of a nebulizer). However, and regardless of the exact choice of delivery mechanism, many of such treatments are based on the use steroids that can have a variety of undesirable side effects.

Where horses are concerned, these increasing rates of occurrence of respiratory conditions such as EA have prompted a consensus in the veterinary community that there is now a pressing need for a similar increase in efforts to discern the causes, and to develop treatments and/or cures. Given the numerous biological similarities between humans and horses, and given the apparent similarities in increases in rates of respiratory conditions over a similar time period for both, a tendency has developed for treatments that were originally created for respiratory conditions in humans to be applied (at least experimentally) to treating respiratory conditions in horses. Unfortunately, this has also created at least the risk of having a similar variety of undesirable side effects in horses, such as those presented by the use of steroids.

An approach is needed to control the healing process for at least musculoskeletal and respiratory tissues to cause the healing process to proceed, as it should, with a better balance between the growth of new respiratory tissues and the “sculpting” of both old and new musculoskeletal and respiratory tissues.

### BRIEF SUMMARY

Technologies are described for more efficiently aggregating α2M molecules for use in an allogeneic or xenogeneic manner.

A method for treating a medical condition of a patient with at least Alpha-2 Macroglobulin (α2M) molecules in an allogeneic or xenogeneic manner includes: drawing whole blood from a donor; separating platelet poor plasma (PPP) containing α2M molecules from other components of the whole blood; filtering waste plasma from the PPP to generate an aggregate of the α2M molecules; and administering at least some of the aggregate to the patient via injection or inhalation, wherein the donor and the patient are different individuals.

A kit for treating a respiratory condition of a patient with at least Alpha-2 Macroglobulin (α2M) molecules in an allogeneic or xenogeneic manner includes at least one separator tube, wherein each separator tube of the at least one separator tube includes: an elongate transparent tube that defines an opening at one end that is sealed with a cap that is penetrable to receive whole blood drawn from a donor; and an amount of separator gel disposed within the separator tube to cooperate with a first centrifugal force exerted on the separator tube for a first period of time during a first centrifuging stage to separate platelet poor plasma (PPP) containing α2M molecules from other components of the whole blood. The kit also includes at least one aggregator, wherein each aggregator of the at least one aggregator includes: a filter; a first cylinder defined by a first cylindrical wall having a first end that is configured to be closable with a septum cap that is penetrable to receive the PPP following the first centrifuging stage, and having a second end that is closed with the filter; and a second cylinder defined by a second cylindrical wall having a first end that is closed where the second cylindrical wall narrows to form a conically-shaped end portion, and having a second end that defines an opening that is configured to be coupled to the filter in a manner that causes a first interior volume of the first cylinder and a second interior volume of the second cylinder to be separated by the filter, wherein the filter is configured to cooperate with a second centrifugal force exerted on the aggregator for a second period of time during a second centrifuging stage to filter waste plasma from the PPP to generate an aggregate of α2M molecules. The kit also includes a transfer device, including: a separator tube port configured to receive each separator tube of the at least one separator tube, one at a time, wherein the separator tube port includes at least one hollow needle configured to penetrate the cap of each separator tube to couple the separator tube to a syringe port of the transfer device; and a syringe port configured to receive an end connector of a transfer syringe that is configured to be coupled to a transfer needle, wherein, following the first centrifuging stage and prior to the second centrifuging stage; while each separator tube of the at least one separator tube is coupled to the separator tube port, a plunger of the transfer syringe is operable to withdraw at least some of the PPP from within the separator tube and into the transfer syringe through the transfer device; and following the transfer of PPP from each separator tube of the at least one separator tube, and with the transfer needle coupled to the end connector to penetrate the septum cap of each aggregator of the at least one aggregator, the plunger of the transfer syringe is operable to inject the PPP within the transfer syringe into the at least one aggregator.

### BRIEF DESCRIPTION OF THE DRAWINGS

The disclosure will be better understood and when consideration is given to the drawings and the detailed description which follows. Such description makes reference to the annexed drawings wherein:

FIGS. 1A, 1B, 1C and 1D, together, provide an overview of aspects of differing embodiments of a system and method of aggregating and administering α2M molecules from whole blood in an autologous manner via inhalation.

FIGS. 2A, 2B, 2C, 2D, 2E, 2F, 2G, 2H, 2I, 2J and 2K, together, provide a more detailed presentation of aspects of an example of the aggregation system of FIG. 1B.

FIGS. 3A, 3B, 3C and 3D, together, provide a more detailed presentation of aspects of an example of the aggregation system of FIG. 1D.

FIGS. 4A, 4B, 4C and 4D, together, provide a more details presentation of aspects of an example of the inhalation system of any of FIGS. 1A-D.

FIGS. 5A, 5B and 5C, together, provide a flow chart of an example of the system and method of FIG. 1D.

FIGS. 6A, 6B, 6C and 6D, together, provide a flow chart of an example of the system and method of FIG. 1B.

### DETAILED DESCRIPTION

In the following detailed description, reference is made to the accompanying drawings that form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Broadly speaking, disclosed herein is a system and a method for aggregating α2M molecules from whole blood from a donor, and then administering those α2M molecules to a patient to treat either a musculoskeletal or respiratory condition. More specifically, the aggregation of α2M molecules may entail the use of centrifugation to separate platelet poor plasma from cellular components, followed by the use of a peristaltic pump to filter waste plasma from the PPP to generate an aggregate of α2M molecules. The administration of the aggregate of α2M molecules may entail the preparation and use of a syringe to inject the aggregate into a musculoskeletal structure in an allogeneic or xenogeneic manner, or may entail the preparation and use of a solution of the aggregate with the nebulizer in an allogeneic or xenogeneic manner.

A method for treating a medical condition of a patient with at least Alpha-2 Macroglobulin (α2M) molecules in an allogeneic or xenogeneic manner includes: drawing whole blood from a donor; separating platelet poor plasma (PPP) containing α2M molecules from other components of the whole blood; filtering waste plasma from the PPP to generate an aggregate of the α2M molecules; and administering at least some of the aggregate to the patient via injection or inhalation, wherein the donor and the patient are different individuals.

A kit for treating a respiratory condition of a patient with at least Alpha-2 Macroglobulin (α2M) molecules in an allogeneic or xenogeneic manner includes at least one separator tube, wherein each separator tube of the at least one separator tube includes: an elongate transparent tube that defines an opening at one end that is sealed with a cap that is penetrable to receive whole blood drawn from a donor; and an amount of separator gel disposed within the separator tube to cooperate with a first centrifugal force exerted on the separator tube for a first period of time during a first centrifuging stage to separate platelet poor plasma (PPP) containing α2M molecules from other components of the whole blood. The kit also includes at least one aggregator, wherein each aggregator of the at least one aggregator includes: a filter; a first cylinder defined by a first cylindrical wall having a first end that is configured to be closable with a septum cap that is penetrable to receive the PPP following the first centrifuging stage, and having a second end that is closed with the filter; and a second cylinder defined by a second cylindrical wall having a first end that is closed where the second cylindrical wall narrows to form a conically-shaped end portion, and having a second end that defines an opening that is configured to be coupled to the filter in a manner that causes a first interior volume of the first cylinder and a second interior volume of the second cylinder to be separated by the filter, wherein the filter is configured to cooperate with a second centrifugal force exerted on the aggregator for a second period of time during a second centrifuging stage to filter waste plasma from the PPP to generate an aggregate of α2M molecules. The kit also includes a transfer device, including: a separator tube port configured to receive each separator tube of the at least one separator tube, one at a time, wherein the separator tube port includes at least one hollow needle configured to penetrate the cap of each separator tube to couple the separator tube to a syringe port of the transfer device; and a syringe port configured to receive an end connector of a transfer syringe that is configured to be coupled to a transfer needle, wherein, following the first centrifuging stage and prior to the second centrifuging stage; while each separator tube of the at least one separator tube is coupled to the separator tube port, a plunger of the transfer syringe is operable to withdraw at least some of the PPP from within the separator tube and into the transfer syringe through the transfer device; and following the transfer of PPP from each separator tube of the at least one separator tube, and with the transfer needle coupled to the end connector to penetrate the septum cap of each aggregator of the at least one aggregator, the plunger of the transfer syringe is operable to inject the PPP within the transfer syringe into the at least one aggregator.

It has been found that α2M molecules, a component of blood in a great many creatures (e.g., camels, dogs, horses, humans and pigs) may be used in an autologous manner to treat such areas of damaged tissue to prevent such misperformances of the healing process. More specifically, it has been found that a dose of α2M molecules may be aggregated from a patient's own blood, and then introduced into damaged musculoskeletal or respiratory tissues of that patient by injection or inhalation to prevent such hyperactivity of proteases at that location, and thus promote effective healing thereat.

Apart from such autologous use of α2M molecules (which is the subject of some of the aforementioned earlier applications) experimental work by the inventors in the present application has revealed that α2M molecules may be used in an allogeneic or xenogeneic manner to treat the same variety of conditions involving tissue damage and protease overactivity. In particular, it has been found that α2M molecules collected from one individual serving as a donor of a species (e.g., a human being or other type of creature) may be injected into a joint of another individual of the same species to successfully treat damage to the musculoskeletal structures of that joint, including damage arising from the onset of arthritis. It has also been found that α2M molecules collected from one individual of a species may be provided, via a nebulizer, to another individual of the same species to successfully treat various respiratory conditions in that other individual, including various types of equine asthma (EA).

Medical research and testing are ongoing to explore and better understand the possible compatibility limitations of such use of α2M molecules in an allogeneic or xenogeneic manner. More specifically, the degree to which α2M molecules are transplantable between different individuals of either the same or different species is currently still uncertain. For example, of particular concern in allogeneic use is compatibility among blood types in some species. As is widely known, among human beings, there are just four blood types, of which type O negative is the one blood type that is deemed to be “universal” such that type O negative blood may be transfused into individuals of any of the other three blood types.

However, while there are many aspects of biology and medicine that may be relatively transferrable among different species, aspects of blood type have not proven to be one of them. For example, in a marked contrast to four types for human beings, among horses, it is estimated that there may be as many as 400,000 blood types. Over time, various criteria have been developed for identifying a subset of equine blood types that are able to be deemed to “universal” to a sufficient degree that blood from horses of that subset may be transfused to other horses with at least a greatly reduced likelihood of causing an adverse reaction. However, with so many blood types, it is usually the case that most transfusions of blood between two horses represents a first instance of a transfer of blood between two horses having their two particular blood types.

This situation concerning horse blood types has served to hamper efforts to determine whether there is such a thing as one or more truly universal blood types. As a result, concerns remain over whether it is possible for blood or blood components from one horse fitting the current criteria for being of a “universal” blood type are able to be repeatedly provided to another horse over a relatively lengthy period of time without that other horse developing an adverse immune response to the further provision of blood or blood components from that very same one horse.

In response to such issues as may exist in horses and/or other species, and in addition to the use of appropriate criteria for identifying donors having a universal blood type, a system and method of tagging and tracking may be implemented together with a system and method for aggregating and administering α2M molecules from the donors in an allogeneic or xenogeneic manner. Based on the ongoing medical research and experimental work for each species, a set of rules may be developed for distinguishing between combinations of donors and patients that are deemed to be safe for the transfer of α2M molecules therebetween, and combinations of donors and patients that are deemed to be unsafe for such a transfer.

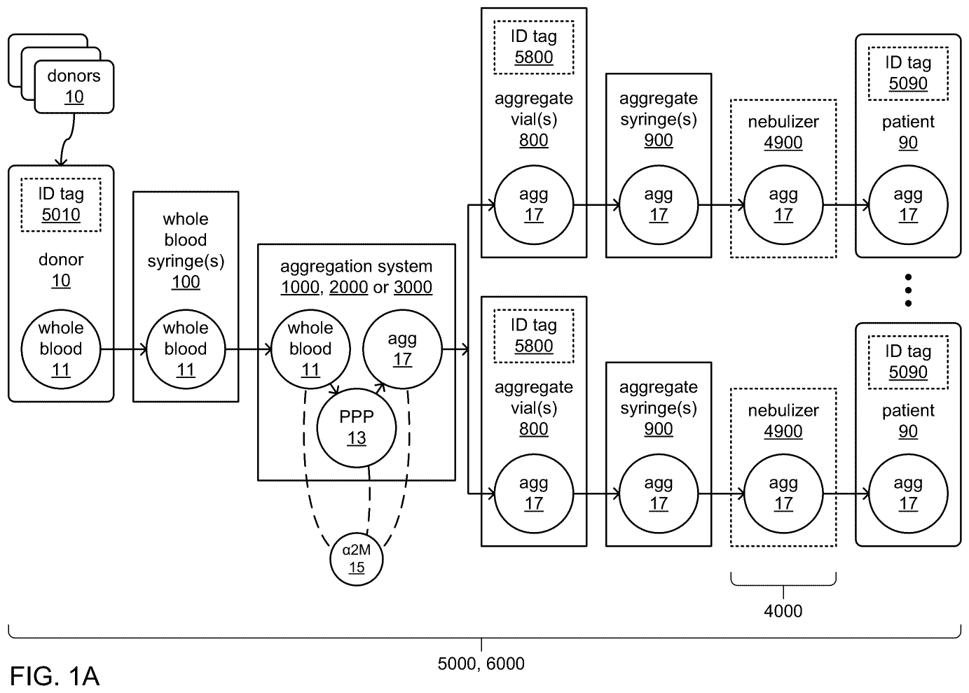

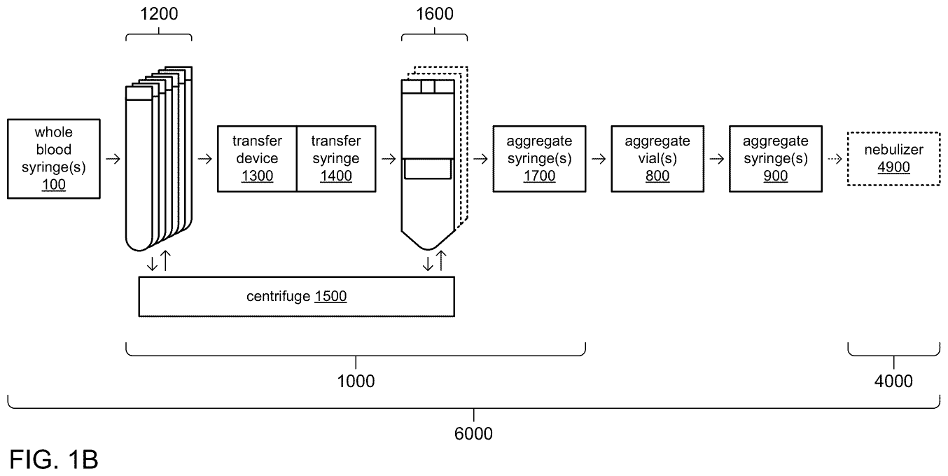

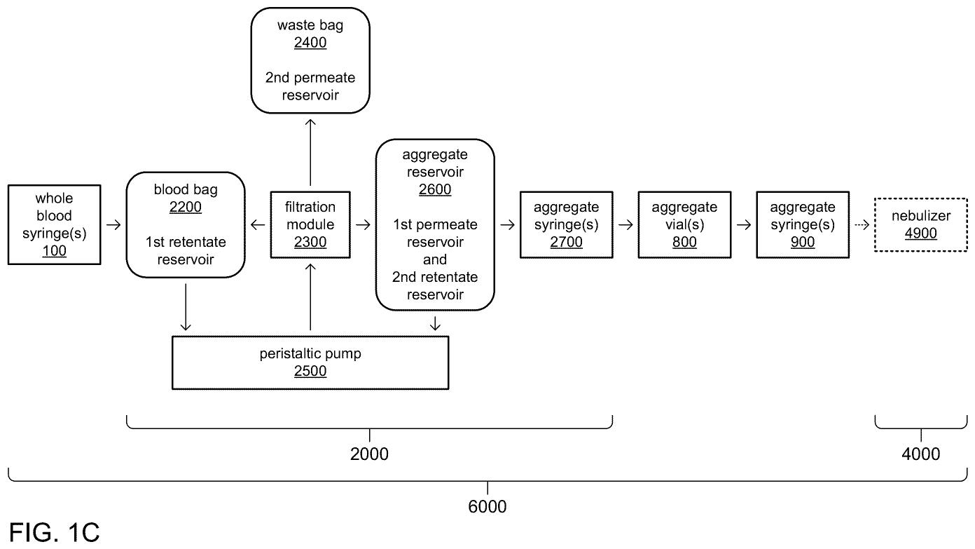

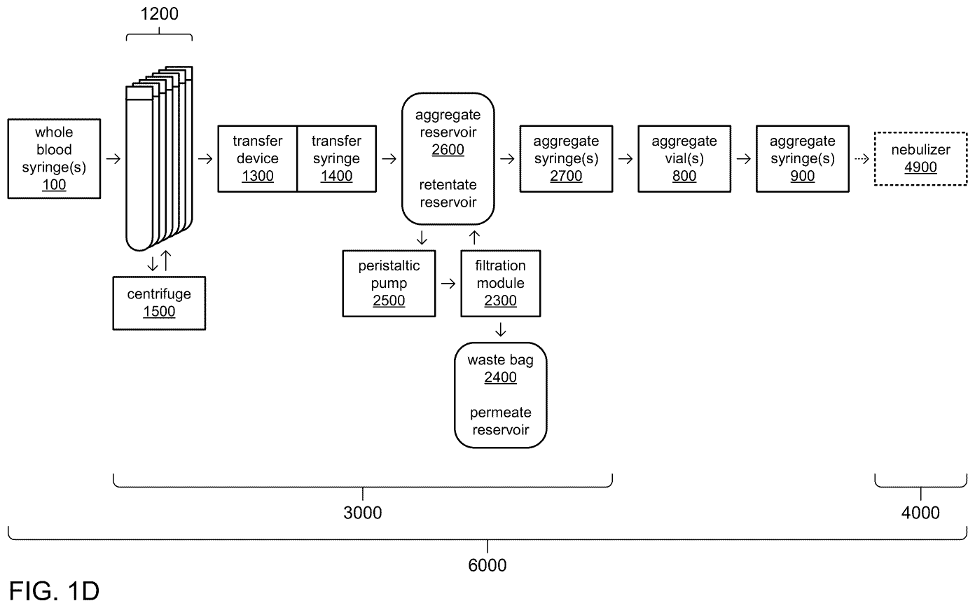

Turning to FIG. 1A, a system 6000 for the aggregation and administration of α2M molecules 15 includes an aggregation system 1000, 2000 or 3000 to perform the aggregation of α2M molecules 15 from whole blood 11. As will be described in greater detail, the aggregation system 1000 may employ a combination of a centrifuge, separator gel and filter to generate an aggregate 17 of the α2M molecules 15 from the whole blood 11; the aggregation system 2000 may employ a combination of a peristaltic pump and multiple filters to do so; and the aggregation system 3000 may employ a hybrid combination of components of the aggregation systems 1000 and 2000 to do so. Regardless of which aggregation system is used, if the aggregate 17 of α2M molecules 15 is administered via inhalation, then the system 6000 may also include an inhalation system 4000 to do so. Also, the system 6000 may additionally include a tracking system 5000 to control the administration function.

Thus, within each aggregation system 1000, 2000 or 3000, various techniques are employed to aggregate α2M molecules 15 from the whole blood 11, and to provide the resulting aggregate of 17 α2M molecules 15 as multiple aliquots in separate ones of a set of multiple aggregate vials 800. As depicted, the generation of the aggregate 17 may entail the generation of a platelet poor plasma (PPP) 13 as an intermediate step. From each aggregate vial 800 in such a set, one or more aggregate syringes 900 and/or a nebulizer 4900 of the inhalation system 4000 may be used in administering the aggregate 17 to the patient 90 via injection or inhalation. As also depicted, it may be that the patient 90 and the aggregate vials 800 are provided with identifier tags 5090 and 5800, respectively, of the tracking system 5000 to enable tracking of at least patients 90 and the aggregate vials 800 from which the aggregate 17 may be administered thereto.

Thus, as depicted, one or more whole blood syringes 100 may be used to draw whole blood 11 from a donor 10 that may be selected from among multiple donors 10, and to provide the whole blood 11 as an input to one of the aggregation systems 1000, 2000 or 3000. It should be noted that it may be that the whole blood 11 of multiple donors 10 is to never be mixed or otherwise combined. Similarly, it may be that the resulting aggregates 17 of α2M molecules 15 of multiple donors 10 are also to never be mixed or otherwise combined. Thus, from each aggregate vial 800, one or more aggregate syringes 900 may be used in administering the aggregate 17 of α2M molecules 15 derived from the whole blood 11 of just a single donor 10 to at least one patient 90.

As also depicted, it may be that the donors 10 are provided with identifier tags 5090 to enable tracking of the identities of the donors 10 and the patients 90. Such tracking may be used, along with a set of rules derived from research and observations of immune responses, to identify and distinguish between safe combinations of donors 10 and patients 90 between which aggregates 17 of α2M molecules 15 may be safely transferred, and unsafe combinations of donors 10 and patients 90 where the risk of an adverse immune response from such a transfer is deemed to be unacceptably high.

FIG. 1B, together with FIG. 1A, provide a high level overview of the aggregation system 1000, and the process of using it. The aggregation of α2M molecules 15 begins with the use of the one or more whole blood syringes 100 to draw an amount of whole blood 11 from a donor 10, and then to transfer portions of that whole blood 11 into each of multiple separator tubes 1200. The set of separator tubes 1200 may then be placed within the centrifuge 1500 to be subjected to centrifugal force for a first predetermined period of time (i.e., a first stage of centrifuging) to cause separation of the PPP 13 containing the α2M molecules 15 from other components of the whole blood 11 (e.g., blood cells and/or cellular components) with the aid of a separator gel incorporated into each of the separator tubes 1200. It should be noted that the PPP 13 may include a variety of other molecules in addition to the α2M molecules 15. In particular, among such other molecules may be serum albumin and/or serotransferrin. Thus, the PPP 13 generated from this first stage of centrifuging may not include solely α2M molecules 15 and blood plasma liquid.

Following such separation of the PPP 13 from the other blood components, one or both of a transfer device 1300 and a transfer syringe 1400 may then be used to retrieve the PPP 13 from each of the separator tubes 1200, and to transfer the PPP 13 to one or more aggregators 1600. The one or more aggregators 1600 may then be placed within the centrifuge 1500 to be subjected to centrifugal force for a second predetermined period of time (i.e., a second stage of centrifuging) to cause filtration of the α2M molecules 15 from the PPP 13 with the aid of a membrane filter incorporated into each aggregator 1600. In this way, the aggregate 17 of the α2M molecules 15 is derived from the PPP 13.

Following such generation of the aggregate 17, one or more aggregate syringes 1700 may then be used to retrieve the aggregate 17 from the one or more aggregators 1600, and to transfer the aggregate 17 to a set of multiple ones of the aggregate vials 800, thereby creating a set of aliquots of the aggregate 17. Following such division of the aggregate 17 into aliquots, the aggregate vials 800 may then be frozen, before being distributed. Such frozen aliquots of the aggregate 17 may then be administered, via injection using just the aggregate syringe(s) 900, or via inhalation using a combination of the aggregate syringe(s) 900 and the nebulizer 4900.

As is about to become apparent, the aggregation system 1000, unlike the aggregation systems 2000 and 3000 that are about to be described, entails the use of relatively small, lightweight and inexpensive components and devices. In particular, unlike the aggregation systems 2000 and 3000 that are about to be described, the aggregation system 1000 does not include a peristaltic pump. Thus, as will be appreciated by those skilled in the art, it is significantly more feasible to bring the components and devices of the aggregation system 1000 on a house call to a farm or other location at which larger animals may be kept, and away from a medical facility. Additionally, the aggregation system 1000 enables the aggregation of α2M molecules 15 from the whole blood 11 of a donor 10 in less time than is possible using either of the aggregation systems 2000 or 3000.

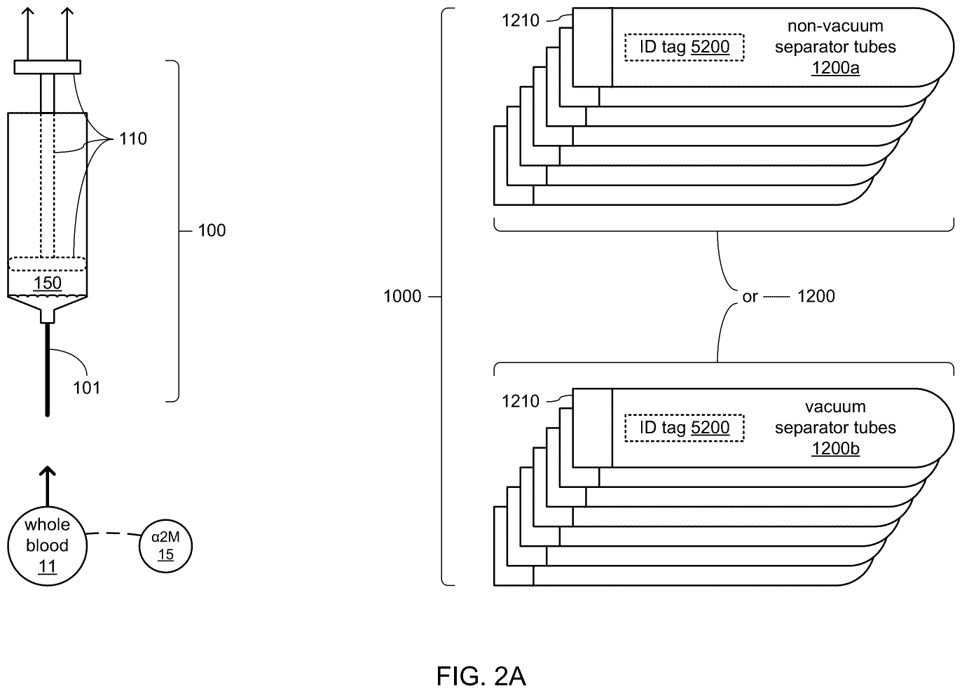

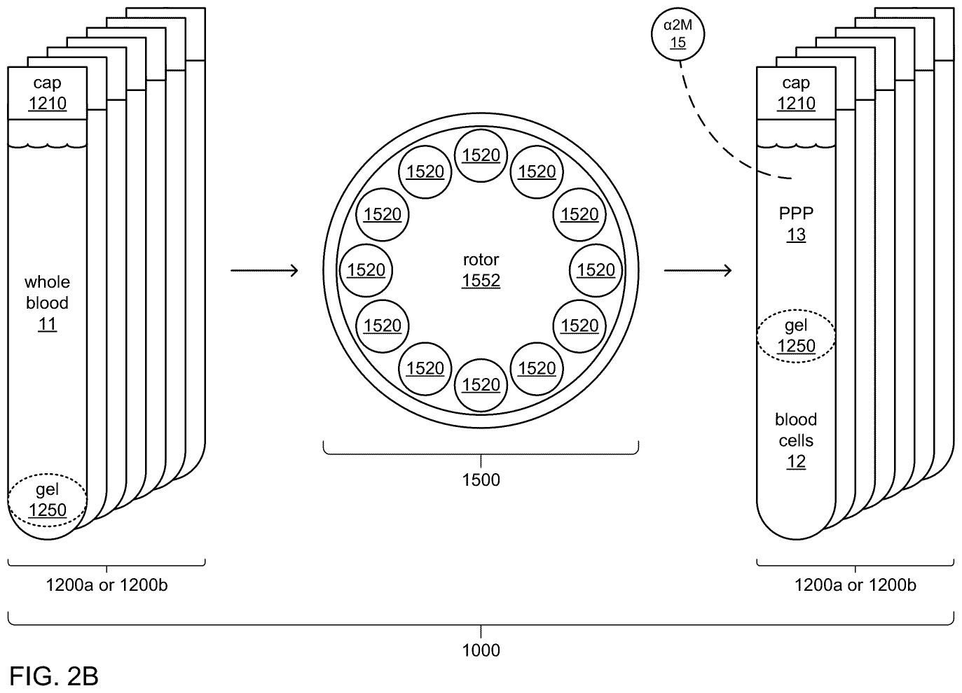

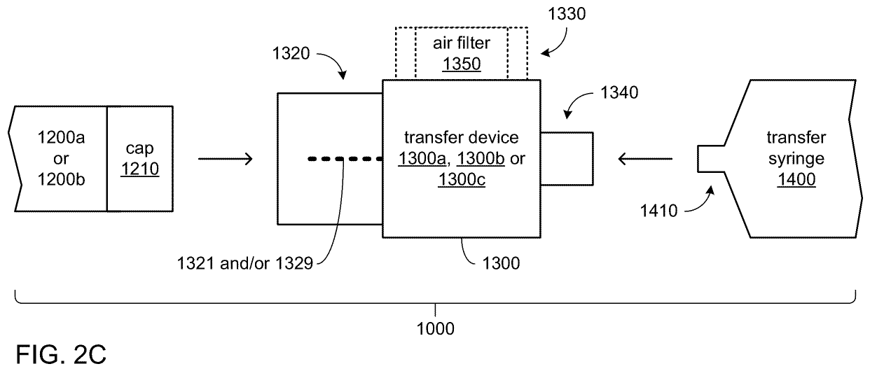

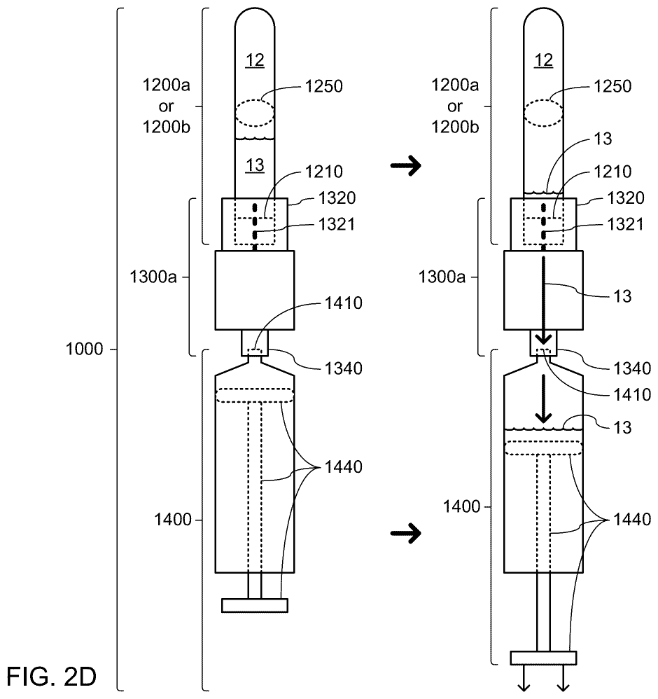

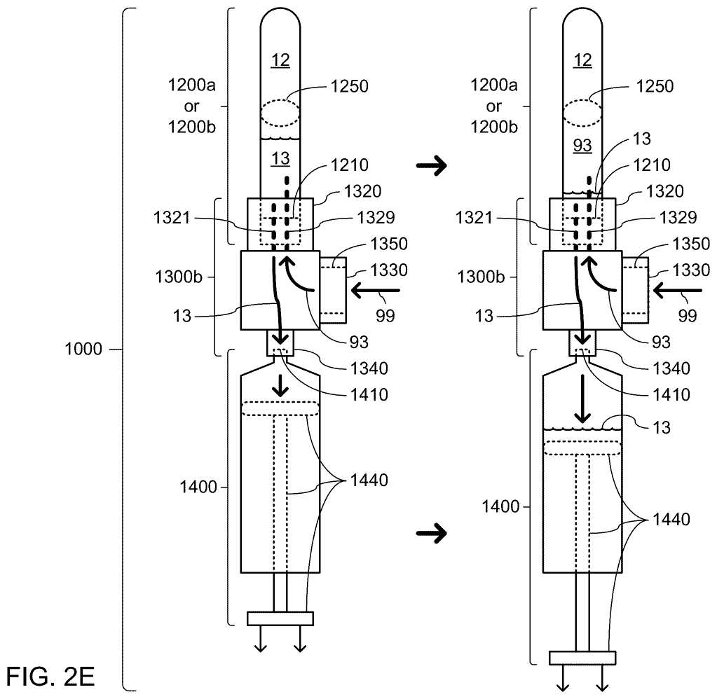

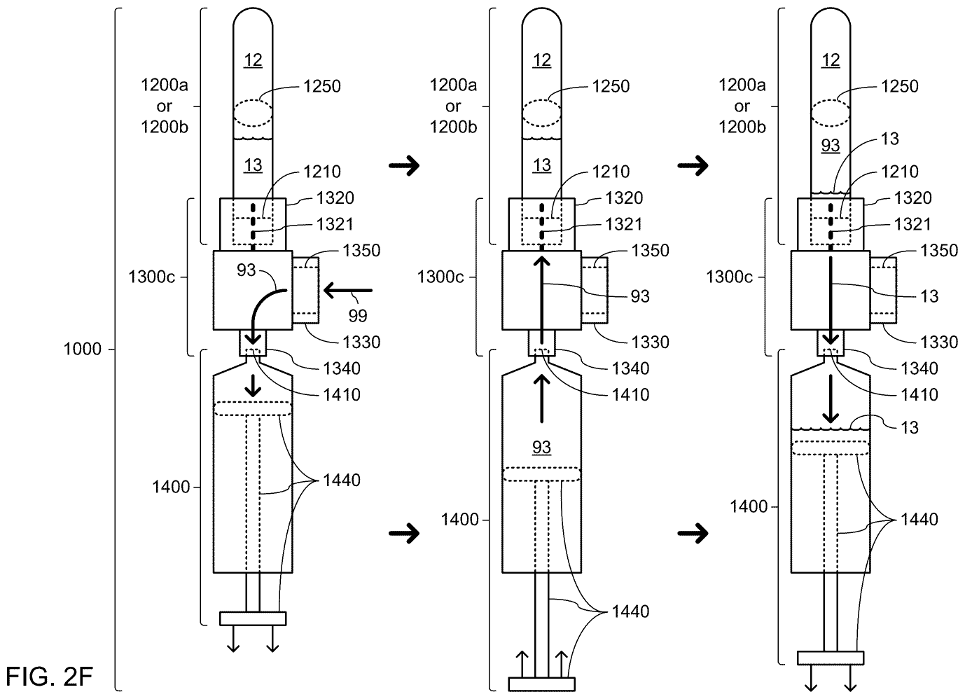

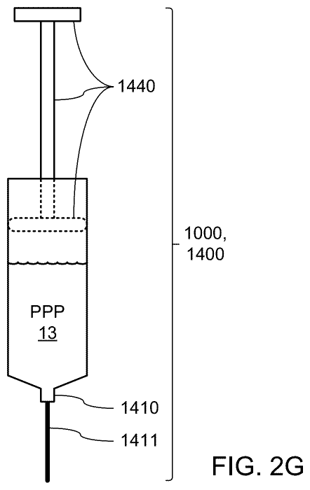

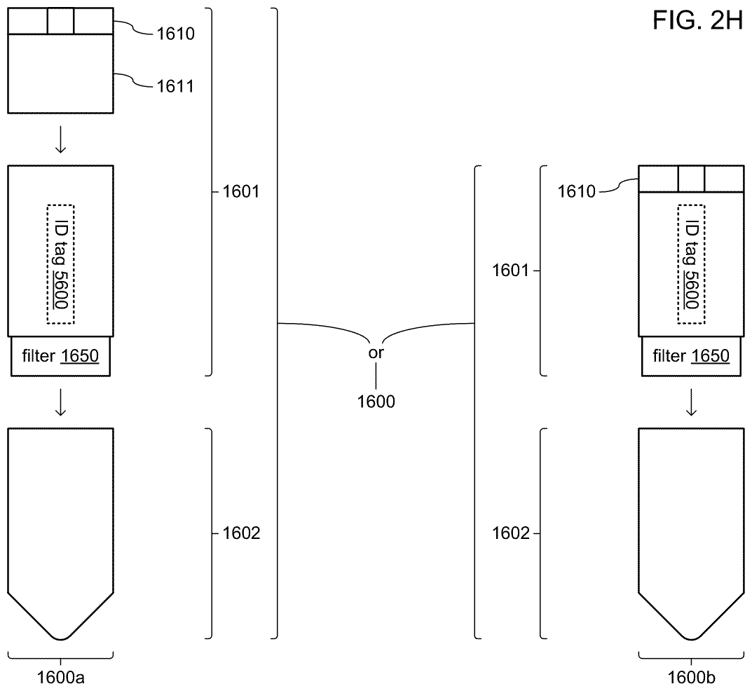

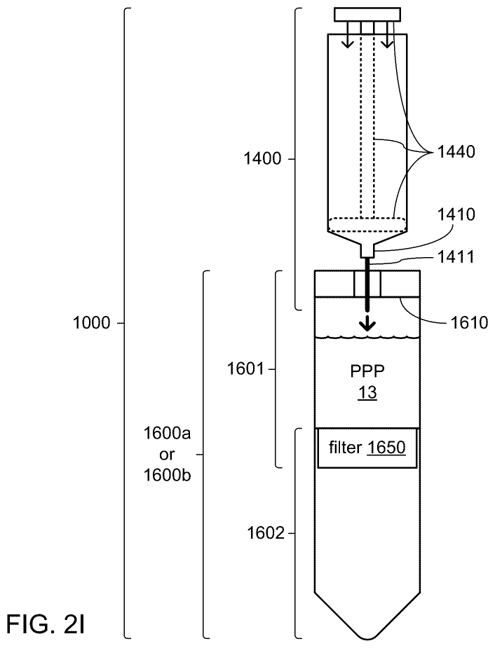

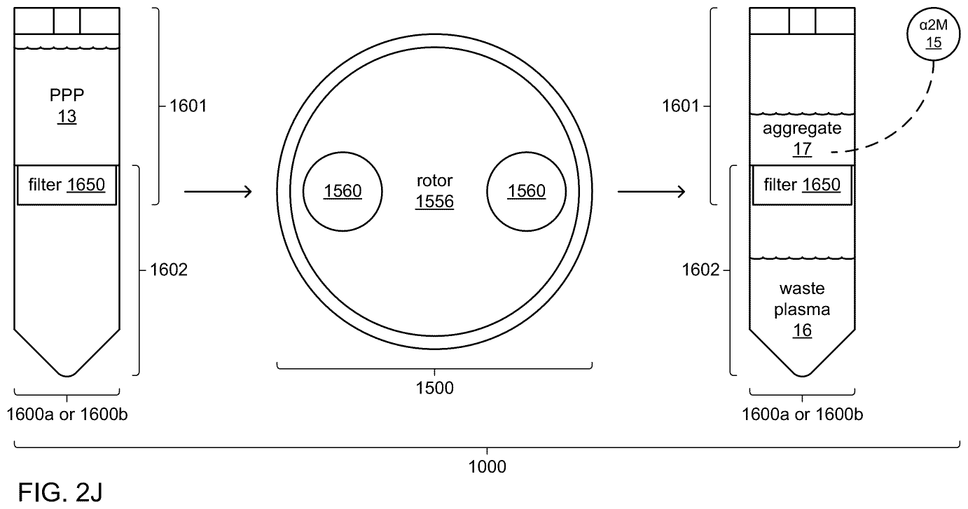

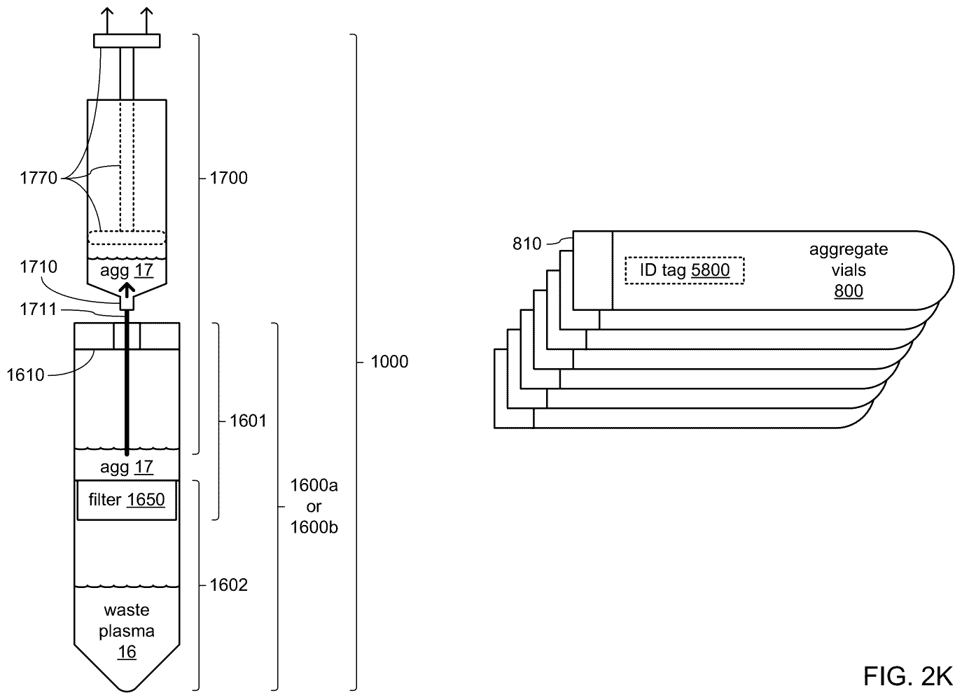

The aggregation system 1000 is depicted and described in greater detail in FIGS. 2A-K.

FIG. 1C, together with FIG. 1A, provide a high level overview of the aggregation system 2000, and the process of using it. The aggregation of α2M molecules 15 begins with the use of the one or more whole blood syringes 100 to draw an amount of whole blood 11 from a donor 10, and then to transfer that whole blood 11 into a blood bag 2200. A set of tubes that interconnect the blood bag 2200, a filtration module 2300, a waste bag 2400, and an aggregate reservoir 2600 may then be fitted to a peristaltic pump 2500. Operation of the peristaltic pump 2500 may squeeze at least one of such tubes in a manner causing flow(s) of whole blood 11 and blood components among the blood bag 2200, the filtration module 2300, the waste bag 2400 and the aggregate reservoir 2600 that brings about the aggregation of the α2M molecules 15 within the aggregate reservoir 2600, thereby generating the aggregate 17 therein.

Following such generation of the aggregate 17, one or more aggregate syringes 2700 may then be used to retrieve the aggregate 17 from the aggregate reservoir 2600, and transfer the aggregate 17 to the set of aggregate vials 800, thereby creating a set of aliquots of the aggregate 17. Again, following such division of the aggregate 17 into aliquots, the aggregate vials 800 may then be frozen, before being distributed. Such frozen aliquots of the aggregate 17 may then be administered, via injection using just the aggregate syringe(s) 900, or via inhalation using a combination of the aggregate syringe(s) 900 and the nebulizer 4900.

FIG. 1D, together with FIG. 1A, provide a high level overview of the aggregation system 3000, and the process of using it. As previously mentioned, the aggregation system 3000 is a hybrid of portions of each of the aggregation systems 1000 and 2000. More specifically, in the aggregation system 3000, the separation of the PPP 13 from other components of the whole blood 11 is performed in a manner similar to how the PPP 13 is separated from other components of the whole blood 11 in the aggregation system 1000. However, the generation of the aggregate 17 from the PPP 13 is performed in a manner similar to how the aggregate 17 is generated in the aggregation system 2000.

Thus, in a manner similar to the aggregation system 1000 of FIG. 1B, the aggregation of α2M molecules 15 begins with the use of the one or more whole blood syringes 100 to draw an amount of whole blood 11 from a donor 10, and then to transfer portions of that whole blood 11 into each of multiple separator tubes 1200. Again, the set of separator tubes 1200 may then be placed within the centrifuge 1500 to be subjected to centrifugal force for a predetermined period of time to cause separation of the PPP 13 from other components of the whole blood 11 with the aid of the separator gel incorporated into each of the separator tubes 1200.

Following the separation of the plasma from the other blood components, and in a manner similar to the aggregation system 2000 of FIG. 1C, one or both of a transfer device 1300 and a transfer syringe 1400 may then be used to retrieve the PPP 13 from each of the separator tubes 1200, and to transfer that plasma to an aggregate reservoir 2600. One of such a set of tubes that interconnect the filtration module 2300, a waste bag 2400, and the aggregate reservoir 2600 may then be fitted to the peristaltic pump 2500. Operation of the peristaltic pump 2500 may squeeze such a tube in a manner causing a flow of the PPP 13 and/or components thereof among the filtration module 2300, the waste bag 2400 and the aggregate reservoir 2600 that brings about the generation of the aggregate 17 within the aggregation reservoir 2600.

Following such generation of the aggregate 17, one or more α2M syringes 2700 may then be used to retrieve the aggregate 17 from the aggregate reservoir 2600, and to transfer the aggregate 17 to the set of aggregate vials 800, thereby creating a set of aliquots of the aggregate 17. Again, following such division of the aggregate 17 into aliquots, the aggregate vials 800 may then be frozen, before being distributed. Such frozen aliquots of the aggregate 17 may then be administered, via injection using just the aggregate syringe(s) 900, or via inhalation using a combination of the aggregate syringe(s) 900 and the nebulizer 4900.

While the aggregation system 1000 may be capable of aggregating α2M molecules 15 from the whole blood 11 of a donor 10 in less time than is possible using either of the aggregation systems 2000 or 3000, the use of a peristaltic pump is more amenable to being scaled up to provide a greater volume of aggregate 17 from each donor 10.

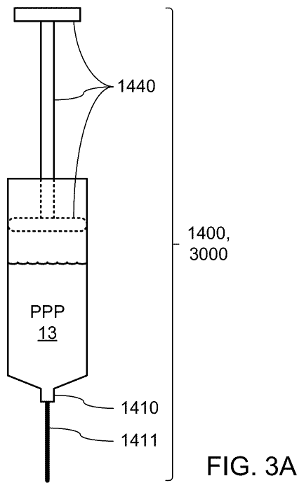

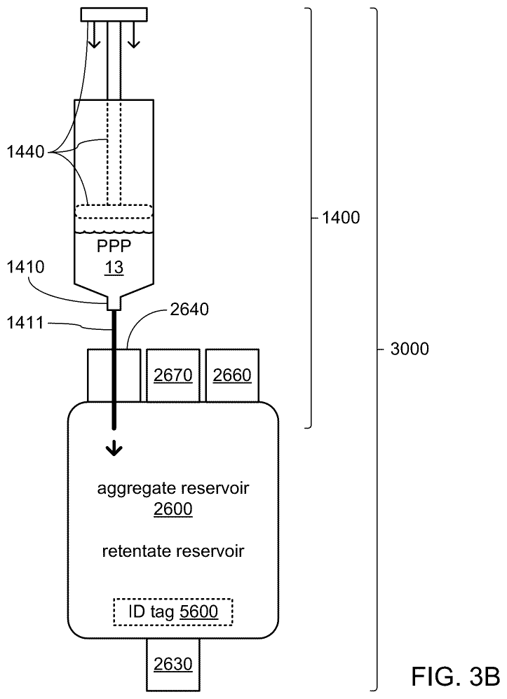

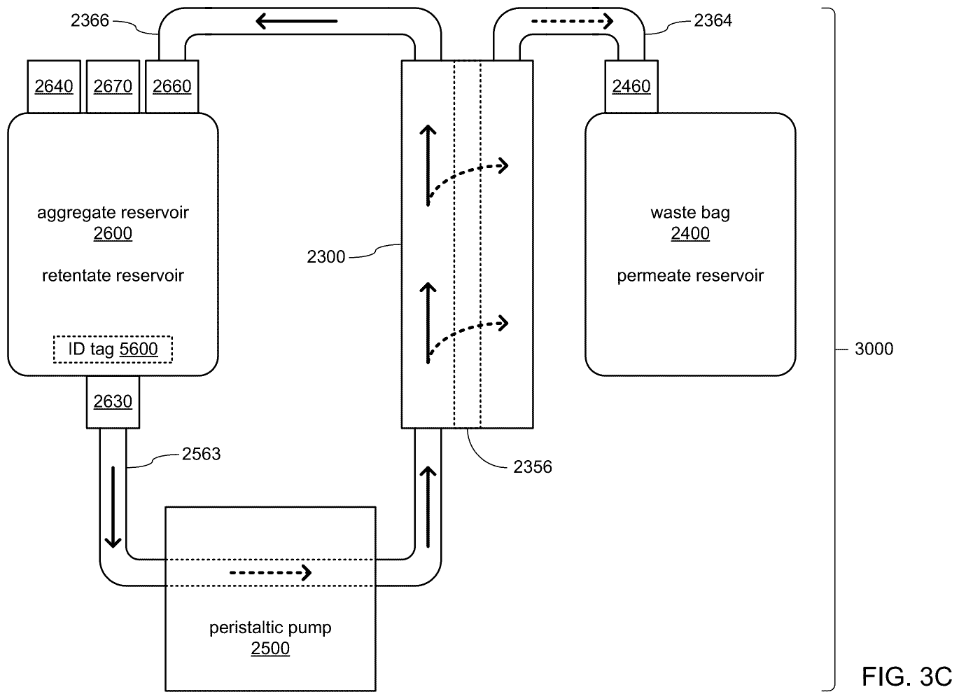

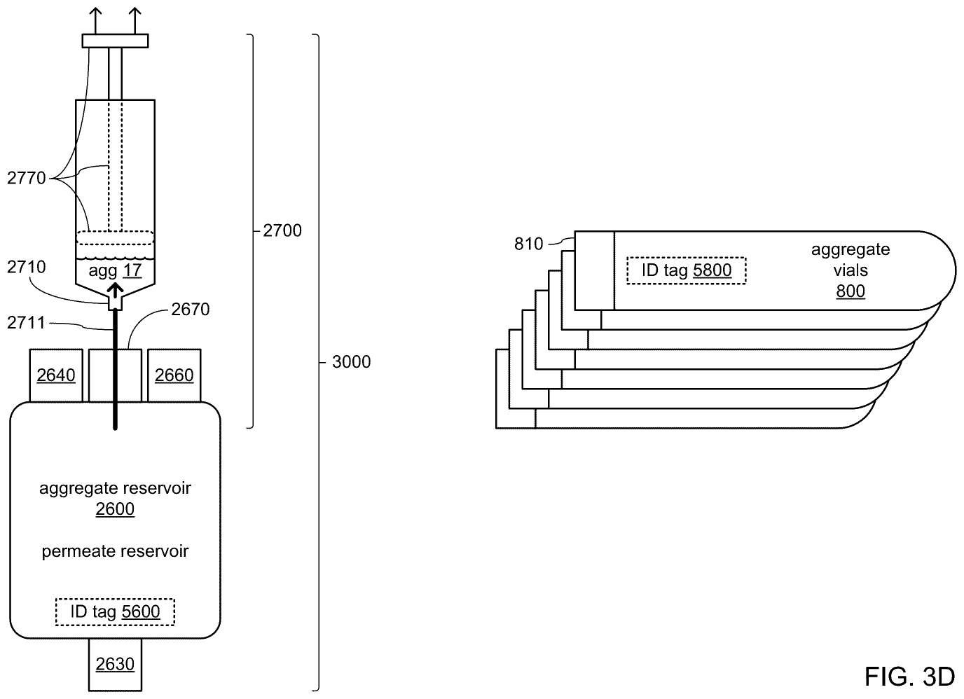

The aggregation system 3000 is depicted and described in greater detail in FIGS. 3A-D.

FIGS. 2A-K, taken together, present aspects of the aggregation system 1000 in greater detail. More specifically, FIGS. 2A-B depict aspects of the separation of PPP 13 containing the α2M molecules 15 from other components of the whole blood 11; FIGS. 2C-I depict aspects of transferring the PPP 13 from the separator tubes 1200 to the aggregator(s) 1600; and FIGS. 2J-2K depict aspects of filtering the PPP 13 to generate the aggregate 17 containing the α2M molecules 15.

Turning to FIG. 2A, as depicted, in the aggregation system 1000, the set of separator tubes 1200 may be either a set of non-vacuum separator tubes 1200a or a set of vacuum separator tubes 1200b. Each of the different types of separator tube 1200a and 1200b may be an elongate transparent tube with a single opening at one end that is sealed with a cap 1210 to at least maintain sterile conditions therein. The cap 1210 may be formed from a relatively flexible material that enables a hollow needle to penetrate therethrough for transferring gases and/or fluids into and/or out of the interior of each of the separator tubes 1200a or 1200b in a manner in which a seal is maintained around such a needle. Such a flexible material may also be self-sealing in a manner that causes a re-sealing of holes formed therethrough by the penetration and subsequent removal of such a needle.

In embodiments of the aggregation system 1000 that include the set of vacuum separator tubes 1200b, each of the vacuum separator tubes 1200b may be a VACUTAINER® tube of a type offered by Becton, Dickson and Company of Franklin Lakes, New Jersey, USA. As will be familiar to those skilled in the art, each such vacuum separator tube 1200b, in its new and unused condition, may be pre-provided with a vacuum therein that the seal provided by the cap 1210 is used to maintain.

Regardless of which of the separator tubes 1200a or 1200b are used, the quantity of separator tubes 1200a or 1200b that are used may vary based on such factors as the volume of whole blood 11 that may be safely drawn from the donor 10, and/or the maximum quantity of separator tubes 1200a or 1200b that may be used with the centrifuge 1500 at a time. As those skilled in the art will readily recognize, the volume of whole blood 11 that may be safely drawn from a donor 10 may depend on at least the species of the donor 10, which again, may include and not be limited to, a camel, a dog, a horse, a human, a pig, etc. Thus, in embodiments of the system 6000 that incorporate the aggregation system 1000, it is contemplated that the system 6000 may be offered in differently-sized variants of kits, such as a smaller variant of kit that may include 1 to 4 separator tubes 1200a or 1200b, a mid-sized variant of kit that may include 5 to 8 separator tubes 1200a or 1200b, and/or a larger variant of kit that may include 9 to 16 (or still more) separator tubes 1200a or 1200b.

A plunger 110 of the whole blood syringe 100 may be operated to draw whole blood 11 containing the α2M molecules 15 from a blood vessel of a donor 10 (whether a human being or other type of creature) and into the whole blood syringe 100 via a needle 101 thereof. The whole blood syringe 100 may include a human-readable scale by which the volume of whole blood that is drawn is able to be measured as the plunger 110 is so operated to ensure that just the amount of whole blood 11 that is needed for the chosen quantity of separator tubes 1200a or 1200b is successfully drawn. After the appropriate volume of whole blood 11 is drawn, the whole blood syringe 100 may then be used to inject a portion of the drawn whole blood 11 into each of the separator tubes 1200a or 1200b through the cap 1210 via the needle 101.

As additionally depicted, in some embodiments, and prior to being used to draw whole blood 11 from a donor 10, the whole blood syringe 100 may be partially pre-filled (e.g., by the nurse, medical technician, veterinarian technician, doctor, veterinarian, etc.) with an amount of an anticoagulent 150, such as a citrate dextrose solution (ACD-A), to prevent the drawn whole blood from coagulating therein.

As also additionally depicted, each of the separator tubes 1200a or 1200b may carry an identifier (ID) tag 5200 that is indicative of identity of the donor 10 to associate the whole blood 11 therein with the individual from which it was drawn. In some embodiments, it may be that the ID tags 5200 are stickers that carry a one-dimensional or two-dimensional bar code that is associated with the donor 10. It may be that such stickers are printed on or about the time that the whole blood 11 is drawn from the donor 10 as part of a procedure that is meant to ensure that each of those particular ID tags 5200 does indeed carry a bar code indicative of the identity of that particular donor 10, and that the set of separator tubes 1200a or 1200b to which those particular ID tags 5200 are applied are indeed caused to contain the whole blood 11 of that particular donor 10. In other embodiments, it may be that the ID tags 5200 are radio frequency identification (RFID) tags that store data serving as an identifier of the donor 10. It may be that such an identifier is caused to be stored within such RFID tags on or about the time that the whole blood 11 is drawn from the donor 10 as part of a procedure that is meant to ensure that each of those particular RFID tags does indeed store an identifier associated with that particular donor 10, and that the set of separator tubes 1200a or 1200b that carry those particular RFID tags is indeed caused to contain the whole blood 11 of that particular donor 10.

Turning to FIG. 2B, each of the different types of separator tube 1200a and 1200b may include (at least in its new and unused condition) a small amount of a separator gel 1250 disposed toward the end opposite the end that is closed with the cap 1210. Thus, as depicted, following the collection and storage of the whole blood 11 among the set of separator tubes 1200a or 1200b, as described above in reference to FIG. 2A, the portion of the whole blood 11 within each of the separator tubes 1200a or 1200b may be disposed therein between the cap 1210 at one end and the separator gel 1250 at the other end.

With the set of separator tubes 1200a or 1200b so filled with portions of whole blood 11, the set of separator tubes 1200a or 1200b may be placed within the centrifuge 1500 to be subjected to centrifugal force for a first period of time that is deemed sufficient to fully separate the PPP 13 containing the α2M molecules 15 from the red and white blood cells 12 thereof. More specifically, and as depicted, the centrifuge 1500 may be used in conjunction with the separator gel 1250 to effect such a separation of components of the whole blood 11. Thus, when such separation of the PPP 13 is complete, the separator gel 1250 within each of the separator tubes 1200a or 1200b should occupy a position that physically separates the PPP 13 from the red and white blood cells 12, thereby preventing these blood components 12 and 13 from becoming mixed together, again. It should again be noted that the PPP 13 may include a variety of other molecules in addition to the α2M molecules 15 (e.g., serum albumin and/or serotransferrin).

As depicted, and as will be familiar to those skilled in the art, the centrifuge 1500 may include a rotor 1552 that defines a set of holding positions 1520 that each have a shape and dimensions selected to hold a tube of matching shape and dimensions, such as one of the separator tubes 1200a or 1200b. As also depicted, it may be that the quantity and placement of such holding positions 1520, as defined by the rotor 1552, may be selected to enable various quantities of such tubes to be distributed among the holding positions 1520 in a manner that distributes the weight thereof in a balanced manner that enables relatively smooth operation of the centrifuge 1500.

As will also be familiar to those skilled in the art, it may be that the depicted rotor 1552 is exchangeable with one or more other rotors to thereby enable the centrifuge 1500 to be reconfigured to work with various different quantities and/or combinations of various tubes and/or other varieties of containers of differing shapes and/or sizes. Alternatively or additionally, it may be that the centrifuge 1500 is fitted with (or otherwise includes) a rotor with 2 or more “buckets.” Each such bucket may be able to be fitted with any of a variety of differing types of holder that may each be designed to provide holding position(s) for a differing quantity of and/or combination of various tubes and/or other varieties of containers of differing shapes and/or sizes.

Turning to FIG. 2C, as depicted, in some embodiments of the aggregation system 1000, the transfer device 1300 may be a single-flow device 1300a. Alternatively, in other embodiments of the aggregation system 1000, the transfer device 1300 may be a dual-flow device 1300b. In still other embodiments of the aggregation system 1000, the transfer device 1300 may be a three-way valve 1300c.

Each of the different types of transfer device 1300a, 1300b and 1300c may incorporate at least the depicted combination of a separator tube port 1320 and a syringe port 1340. Each of the different types of transfer device 1300b and 1300c may additionally incorporate a filtered air port 1330. As is about to be described, each of the different types of transfer device 1300a, 1300b and 1300c is configured to enable the PPP 13 to be transferred from a separator tube 1200a or 1200b coupled to the separator tube port 1320, and to a transfer syringe 1400 coupled to the syringe port 1340. Additionally, and as is also about to be described, each of the different types of transfer device 1300b and 1300c is additionally configured to also enable external air surrounding the transfer device 1300b or 1300c to be drawn in through an air filter 1350 at the filtered air port 1330, and conveyed to the separator tube 1200a or 1200b that is coupled to the separator tube port 1320.

It is envisioned that the interior volume of the transfer syringe 1400 is sufficiently large that all of the sum total of the amounts of the PPP 13 separated within all of the separator tubes 1200a or 1200b (as a result of being subjected to centrifugal force by the centrifuge 1500, as earlier described) is able to be combined and retained within the transfer syringe 1400. As a result, it is envisioned that the transfer syringe 1400 is to remain connected to the syringe port 1340 by its end connector 1410 throughout the time that the PPP 13 is being transferred from each of the separator tubes 1200a or 1200b, and into the transfer syringe 1400.

FIG. 2D depicts aspects of the manner in which the single-flow device 1300a enables a transfer of PPP 13 out of the separator container 1200a or 1200b, and into the transfer syringe 1400. As depicted, the separator tube port 1320 may incorporate a PPP needle 1321 that is positioned to penetrate through the cap 1210 of a separator tube 1200a or 1200b to enable the flow through each of gases and/or liquids out of such a separator tube 1200a or 1200b. As also depicted, the syringe port 1340 may be configured to form a connection with an end connector 1410 carried at one end of the transfer syringe 1400.

As depicted, with a separator tube 1200a or 1200b coupled to the separator tube port 1320 such that the PPP needle 1321 penetrates the cap 1210 thereof, and with the end connector 1410 of the transfer syringe 1400 coupled to the syringe port 1340, there may be an initial equalization of pressures thereamong. However, it has been discovered that the operation of the single-flow transfer device 1300a is not significantly changed by whether non-vacuum separator tubes 1200a or vacuum separator tubes 1200b are coupled to the separator tube port 1320. Thus, regardless of which type of separator tube 1200a or 1200b is coupled to the separator tube port 1320, pulling the plunger 1440 of the transfer syringe 1400 in a direction away from the end connector 1410 thereof may draw PPP 13 from within the separator tube 1200a or 1200b, and into the transfer syringe 1440, via the PPP needle 1321 and the end connector 1410.

FIG. 2E depicts aspects of the manner in which the dual-flow device 1300b enables a simultaneous transfer of filtered air 93 into a separator container 1200a or 1200b, and of PPP 13 out of the separator container 1200a or 1200b as part of transferring PPP 13 to the transfer syringe 1400. As depicted, the separator tube port 1320 may incorporate both an air needle 1329 and a PPP needle 1321 that are each positioned to penetrate through the cap 1210 of a separator tube 1200a or 1200b to enable the flow through each of gases and/or liquids into and/or out of such a separator tube 1200a or 1200b. Again, the syringe port 1340 may be configured to form a connection with an end connector 1410 carried at one end of the transfer syringe 1400.

As depicted, with a separator tube 1200a or 1200b coupled to the separator tube port 1320 such that the needles 1321 and 1329 penetrate the cap 1210 thereof, and with the end connector 1410 of the transfer syringe 1400 coupled to the syringe port 1340, there may be an initial equalization of pressures thereamong. More specifically, and especially where a vacuum separator tube 1200b is coupled to the separator tube port 1320, external air 99 may be drawn into the dual-flow device 1300a through the air filter 1350 of the filtered air port 1330, and then the resulting filtered air 93 may be conveyed into a separator tube 1200a or 1200b at the separator tube port 1320 via the air needle 1329. Pulling the plunger 1440 of the transfer syringe 1400 in a direction away from the end connector 1410 thereof may then draw PPP 13 from within the separator tube 1200a or 1200b, and into the transfer syringe 1440, via the PPP needle 1321 and the end connector 1410. In turn, more filtered air 93 may be drawn into the separator tube 1200a or 1200b to replace the PPP 13 that is so drawn out.

FIG. 2F depicts aspects of the manner in which the three-way valve 1300c enables a selective transfer of filtered air 93 into a separator container 1200a or 1200b, and of PPP 13 out of the separator container 1200a or 1200b as part of transferring the PPP 13 to the transfer syringe 1400. As depicted, the separator tube port 1320 of the three-way valve 1300c may incorporate just a single PPP needle 1321 that is positioned to penetrate through the cap 1210 of a separator tube 1200a or 1200b to enable the flow therethrough of gases and/or liquids into and/or out of such a separator tube 1200a or 1200b. Again, the syringe port 1340 may be configured to form a connection with an end connector 1410 carried at one end of the transfer syringe 1400.

The three-way valve 1300c may incorporate a manually-operable valve (not specifically shown) of a type that is operable between at least two positions, where each position of the at least two positions causes one of the three ports 1320, 1330 or 1340 to be closed off from the other two of these two ports, while allowing gases and/or liquids to flow freely between the other two.

As depicted, for each separator tube 1200a or 1200b that is connected to the separator tube port 1320, the transfer of PPP 13 therefrom, and into the transfer syringe 1400, may begin with the three-way valve 1300c being operated to close off the separator tube port 1320, thereby connecting the syringe port 1340 to the filtered air port 1330. With the separator tube port 1320 so closed off, the plunger 1440 of the transfer syringe 1400 may be operated to draw filtered air 93 into the transfer syringe 1400. More precisely, the plunger 1440 of the transfer syringe 1400 may be operated to cause external air 99 that surrounds the three-way valve 1300c to be drawn in through the air filter 1350, thereby being filtered to become the filtered air 93 that is drawn into the transfer syringe 1400.

With an amount of such filtered air 93 now within the transfer syringe 1400, the three-way valve 1300c may then operated to close off the filtered air port 1330, thereby connecting the syringe port 1340 to the separator tube port 1320. With the filtered air port 1330 so closed off, the plunger 1440 of the transfer syringe 1400 may be operated to send filtered air 93 out of the transfer syringe 1400, through the three-way valve 1300c, through the PPP needle 1321, and into the separator tube 1200a or 1200b that is coupled to the separator tube port 1320. With filtered air 93 so conveyed into the separator tube 1200a or 1200b, the plunger 1440 of the transfer syringe 1400 may then be operated to draw most, if not all, of the PPP 13 out of the separator tube 1200a or 1200b, through the PPP needle 1321, through the three-way valve 1300c, and into transfer syringe 1400.

Referring back to each of FIGS. 2D-F, it should be noted, that such transfers of PPP 13 from the separator tubes 1200a or 1200b, and into the transfer syringe 1400 may need to be performed with the depicted combination of the separator tube 1200a or 1200b, the transfer device 1300a/1300b/1300c, and the transfer syringe 1400 held in an orientation in which the separator tube 1200a or 1200b is at a higher elevation than the transfer syringe 1400.

Turning FIG. 2G, following the transfer of PPP 13 out of each of the separator tubes 1200a or 1200b, and into the transfer syringe 1400, the transfer syringe 1400 may then be disconnected from the transfer device 1300a, 1300b or 1300c. Then, a transfer needle 1411 may be connected to the end connector 1410 of the transfer syringe 1400 in preparation for injecting the PPP 13 into an aggregator 1600.

Turning to FIG. 2H, as depicted, the aggregator 1600 may include a combination of a first cylinder 1601 and a second cylinder 1602. Both of these cylinders 1601 and 1602 may be of a generally elongate shape such that each defines a pair of ends.

One end of the first cylinder 1601 may be sealed (or sealable) with a septum cap 1610 that may provide a self-sealing aperture through which a needle or other form of tube of relatively small diameter tube may be inserted to effect the transfer of gases and/or liquids into and/or out of the interior volume of the first cylinder 1601. The other end of the first cylinder 1601 may incorporate a membrane filter 1650. In some embodiments, the membrane filter 1650 may have a molecular weight cutoff ranging from 100 kD to 500 kD.

The second cylinder 1602 may be configured to make the aggregator 1600 more amenable for use with the centrifuge 1500. More specifically, one end of the second cylinder 1602 may be closed off with a conical end to ease insertion into the centrifuge 1500, while the other end may be open to enable the two cylinders 1601 and 1602 to be assembled by inserting part of the end of the first cylinder 1601 that includes the membrane filter 1650 therein.

It should be noted (and as depicted) that, in some embodiments, the aggregator 1600 may be of an extended length variant 1600a in which the volume of the first cylinder 1601 is increased by sealing the end opposite the membrane filter 1650 with an extended variant of the septum cap 1610 that provides a cylindrical extension 1611 to the cylindrical wall of the first cylinder 1601 to increase the length of the first cylinder 1601. Alternatively, in other embodiments, the aggregator 1600 may be of a standard length variant 1600b in which the volume of the first cylinder 1601 is not so increased. More precisely, instead of sealing the end opposite the membrane filter 1650 with the extended variant of the septum cap 1610, a standard variant of the septum cap 1610 is used in the standard length variant 1600b that does not provide the cylindrical extension 1611 of the extended length variant 1600a.

As additionally depicted, the aggregator 1600 may carry an ID tag 5600 that is indicative of identity of the donor 10. In a manner similar to the ID tags 5200, it may be that the ID tag 5600, regardless of the technology it is based upon, may be printed and/or caused to store an identifier at a time on or about the time that the whole blood 11 is drawn from the donor 10. In this way, like the ID tags 5200, the ID tag 5600 is caused to store an indication of the identity of the individual from which the whole blood 11 is drawn.

Turning to FIG. 2I, with the aggregator 1600a or 1600b assembled, and with the transfer needle 1411 connected to the end connector 1410 of the transfer syringe 1400, the transfer needle 1411 may then be inserted through the aperture of the septum cap 1610. The plunger 1440 of the transfer syringe 1400 may then be operated to transfer the PPP 13 out of the transfer syringe 1400, and into the first cylinder 1601 through the transfer needle 1411 and the aperture of the septum cap 1610.

Turning to FIG. 2J, after the PPP 13 has been transferred into the first cylinder 1601, the still assembled aggregator 1600 may then be placed within the centrifuge 1500 to be subjected to centrifugal force for a second period of time that is deemed sufficient to filter out waste plasma 16 from the PPP 13, thereby deriving an aggregate 17 of α2M molecules 15 from the PPP 13. Thus, when such filtration is complete, the aggregate 17 of α2M molecules 15 should remain within the first cylinder 1601, while other components of the PPP 13 should be retained within the second cylinder 1602 as the depicted waste plasma 16. It should again be noted that the aggregate 17 may include a variety of other molecules in addition to the α2M molecules 15 (e.g., serum albumin and/or serotransferrin).

In a manner similar to what was discussed in reference to FIG. 2B, the centrifuge 1500 may include a rotor 1556 that defines a pair of holding positions 1560 that each have a shape and dimensions selected to hold a tube of matching shape and dimensions, such as the aggregator 1600. As also depicted, it may be that the pair of such holding positions 1560, as defined by the rotor 556, may be positioned to enable the placement of a pair of the aggregators 1600 at locations that distribute the weight thereof in a balanced manner that enables relatively smooth operation of the centrifuge 1500.

It should be noted that, although a variant of the rotor 1556 that provides a pair of the holding positions 1560 is depicted and described herein, other embodiments are possible in which the rotor 1556 may have more or fewer of such holding positions 1560. Further, to address situations in which the centrifuge is to be operated with a variant of the rotor 1556 that includes a quantity of holding positions 1560 that differ from the quantity of aggregators 1600 that are to be inserted therein, one or more dummy weights of a shape, size and/or weight similar to an aggregator 1600 may be used to enable balancing of the centrifuge 1500. Alternatively, an extra aggregator 1600 filled with water may be used to serve such a purpose.

As will also be familiar to those skilled in the art, it may be that the depicted rotor 1556 is exchangeable with one or more other rotors (e.g., the rotor 1552 of FIG. 2B) to thereby enable the centrifuge 1500 to be reconfigured to work with various different quantities and/or combinations of various tubes, aggregators and/or other varieties of containers of differing shapes and/or sizes. Alternatively or additionally (and as was earlier discussed in reference to FIG. 2B), it may be that the centrifuge 1500 is fitted with (or otherwise includes) a rotor with 2 or more “buckets.” Each such bucket may be able to be fitted with any of a variety of differing types of holder that may each be designed to provide holding position(s) for a differing quantity of and/or combination of various tubes and/or other varieties of containers of differing shapes and/or sizes.

It should be noted that such use of the aggregator 1600 with the centrifuge 1500 to perform the filtration to derive the aggregate 17 from the PPP 13 originally transferred into the first cylinder 1601 has been found to provide a simpler approach than a peristaltic pump. The centrifuge 1500 is also a far simpler and far less expensive piece of equipment than a peristaltic pump. As a result, the aggregation system 1000 may be more suitable for being carried by and/or installed within a vehicle used to make house calls.

Turning to FIG. 2K, following such derivation of the aggregate 17, the aggregate 17 may be transferred to the set of aggregate vials 800 via the one or more aggregate syringes 1700, thereby defining the set of aliquots of the aggregate 17 derived from the whole blood 11 drawn in FIG. 2A. More specifically, a needle 1711 connected to an end connector 1710 of each of the one or more aggregate syringes 1700 may be inserted through the aperture of the septum cap 1610. With the needle 1711 of each of the one or more aggregate syringes 1700 so inserted, a plunger 1770 thereof may then be operated to retrieve the aggregate 17 from within the first cylinder 1601, and to subsequently transfer the aggregate 17 to the set of aggregate vials 800. Where more than one aggregator 1600 is used to derive the aggregate 17, such operations may be repeated to transfer the aggregate 17 out of each such aggregator 1600.

It is envisioned that the amount of the aggregate 17 that is aggregated from the whole blood 11 originally drawn from the donor 10 will be more than enough to fill more than one of the aggregate vials 800. In some embodiments, it may be deemed desirable to provide an amount within each of the aggregate vials 800 that is large enough to support multiple administrations of a dose, or of multiple doses, to the patient 90 (e.g., multiple administrations over the course of one or more hours). However, in other embodiments, it may be deemed desirable to provide an amount within each of the aggregate vials 800 that is small enough to support just a single dose or single administration of a dose to the patient 90, thereby enabling each dose or administration of a dose to be kept frozen in a separate aggregate vial 800 until the time comes that it is needed. Thus, it is envisioned that each aliquot is to be sized to enable one of the aliquots to be used to deliver a first amount to the patient 90, while one or more other aliquots may remain stored in a freezer to be preserved for the later instances of delivery to the same patient 90. In this way, the provision of multiple administrations and/or doses over the course of multiple days, weeks, months, etc. may be more easily supported.

As additionally depicted, each of the vials 800 may carry an ID tag 5800 that is indicative of identity of the donor 10. In a manner similar to the ID tags 5200 and 5600, it may be that the ID tags 5800, regardless of the technology on which they are based, may be printed and/or caused to store an identifier at a time on or about the time that the whole blood 11 is drawn from the donor 10.

FIGS. 3A-D, taken together, present aspects of the aggregation system 3000 in greater detail. As previously discussed, the aggregation system 3000 may be a hybrid that combines a portion of the aggregation system 1000 in which a centrifuge 1500 is used to separate PPP 13 containing α2M molecules 15 from other components of whole blood 11 (depicted in detail in FIGS. 2A-B), and a portion of the aggregation system 2000 in which filtration (based on a peristaltic pump 2500) is used to derive aggregate 17 of the α2M molecules 15 from the PPP 13 (depicted in FIG. 1C). Thus, FIG. 3A presents the results of having separated the PPP 13 from other components of the whole blood 11 drawn from a donor 10; and FIGS. 3B-3D depict aspects of using filtration to derive the aggregate 17.

Turning to FIG. 3A, in the aggregation system 3000, the PPP 13 containing the α2M molecules 15 may be separated from other components of the whole blood 11, and may be transferred to the depicted transfer syringe 1400 in a manner similar to what has already been depicted and discussed in reference to FIGS. 2A-2F concerning the aggregation system 1000.

Turning to FIG. 3B, the transfer syringe 1400 filled with the PPP 13 may be connected to the aggregate reservoir 2600, and the plunger 1440 thereof may be operated to inject the PPP 13 into the aggregate reservoir 2600.

The aggregate reservoir 2600 may be a bag formed of flexible material, and may incorporate multiple ports by which needles of syringes may be connected thereto to inject fluids into the aggregate reservoir 2600 and/or to withdraw fluids therefrom. Such ports may also enable ends of various flexible tubes to be connected to the aggregate reservoir 2600 to enable flows of gas and/or liquid into and/or out the aggregate reservoir 2600. More specifically, the aggregate reservoir 2600 may incorporate a filling port 2640 through which the transfer syringe 1400 may provide the PPP 13, as just described, and an extraction port 2670 from which the aggregate 17 of the α2M molecules 15 may later be extracted, as will be described.

Turning to FIG. 3C, the aggregate reservoir 2600 may also incorporate an output port 2630 to which one end of a flexible output tube 2563 may be connected, with the other end thereof being connected to a portion of a filter module 2300 that includes a cross-flow filter 2356. The aggregate reservoir 2600 may further incorporate a retentate port 2660 to which one end of a flexible retentate tube 2366 may be connected, with the other end thereof being connected to the same portion of the filter module 2300 to which the output tube 2563 is connected.

As will be familiar to those skilled in the art, the peristaltic pump 2500 may incorporate a rotary component (not specifically shown) that carries multiple rollers configured to repeatedly squeeze a flexible tube in a manner that moves along a portion of its length to thereby cause a flow of fluid therealong. An advantage of the peristaltic pump 2500 is that the fluid is caused too flow in a manner in which it is never directly exposed to any component of the peristaltic pump 2500, thereby simplifying efforts to maintain sanitary and non-contaminant conditions for the fluid. In this way, the need to clean components of the peristaltic pump 2500, itself, due to direct contact with the fluid is entirely obviated, as is the need to manufacture components of the peristaltic pump 2500 from materials specifically chosen to not chemically interact with and/or otherwise contaminate the fluid. Peristaltic pumps have long been favored for use in pumping blood and/or other bodily fluids for at least these reasons.

Thus, a portion of the output tube 2563 extending from the output port 2630 of the α2M reservoir 2600, and to the filter module 2300, may be inserted into the portion of the peristaltic pump 2500 that incorporates such a rotary component to cause the output tube 2563 to be used in pumping the PPP 13 from the aggregate reservoir 2600, and toward the cross-flow filter 2356.

As will be familiar to those skilled in the art, the cross-flow filter 2356 may be physically shaped and/or otherwise configured to direct a flow of fluid in a tangential direction relative to a face of its filter media such that the flow of fluid essentially flows cross-wise relative to that face of its filter media. In some embodiments, it may be that the filter media of the cross-flow filter 2356 is selected to prevent molecules larger in weight than 500 kDa from passing therethrough.

With the output tube 2563 inserted into a portion of the peristaltic pump 2500, and with the peristaltic pump 2500 operated to cause a flow of the PPP 13 out of the aggregate reservoir 2600 and toward the cross-flow filter 2356, the PPP 13 from the aggregate reservoir 2600 may be caused to flow tangentially across the face of the filter media of the cross-flow filter 2356. As a result, molecules of the PPP 13 that are small enough to permeate through the filter media of the cross-flow filter 2356 are allowed to do so, and are caused by the pressure of the pumping of the PPP 13 into the filter module 2300 (and through the cross-flow filter 2356) to leave the filter module 2300 via a permeate tube 2364, and to be deposited within a waste bag 2400 via a permeate port 2460 thereof. At the same time, components of the PPP 13 that do not permeate the filter media of the cross-flow filter 2356 are caused by the same pressure to leave the filter module 2300 via the retentate tube 2366, and to be returned to the aggregate reservoir 2600 via the retentate port 2660 thereof.

Like the aggregate reservoir 2600, the waste bag 2400 may also be formed of flexible material. The waste bag 2400 may also incorporate at least the aforementioned permeate port 2460 by which one end of the permeate tube 2364 may be connected thereto to enable at least a flow of gas and/or liquid into the waste bag 2400.

In this manner, the PPP 13 within the aggregate reservoir 2600 is able to be repeatedly circulated in a cross-wise manner relative to the filter media of the cross-flow filter 2356 until at least most, if not all, of the components of the PPP 13 that are able to permeate the filter media have done so. As a result, the aggregate reservoir 2600 is caused, over time, to contain the aggregate 17 of the α2M molecules 15, while other components of the PPP 13 are filtered therefrom, and deposited within the waste bag 2400.

As also additionally depicted, the aggregate reservoir 2600 may carry an identifier (ID) tag 5600 that is indicative of identity of the donor 10. Again, in some embodiments, it may be that the ID tag 5600 is a sticker that carries a one-dimensional or two-dimensional bar code that is associated with the donor 10. It may be that such a sticker is printed on or about the time that the whole blood 11 is drawn from the donor 10 as part of a procedure that is meant to ensure that the ID tag 5600 does indeed carry a bar code indicative of the identity of the donor 10, and that the aggregate reservoir 2600 to which the ID tag 5600 is applied is indeed caused to contain the PPP 13 derived from the whole blood 11 of that particular donor 10. In other embodiments, it may be that the ID tag 5600 is a radio frequency identification (RFID) tag that stores data serving as an identifier of the donor 10. It may be that such an identifier is caused to be stored within such an RFID tag on or about the time that the whole blood 11 is drawn from the donor 10 as part of a procedure that is meant to ensure that the RFID tag does indeed store an identifier associated with that particular donor 10, and that the aggregate reservoir 2600 that carries the RFID tag is indeed caused to contain the PPP 13 derived from the whole blood 11 of that particular donor 10.

Turning to FIG. 3D, following such aggregation of the α2M molecules 15 to generate the aggregate 17 of the α2M molecules 15, the aggregate 17 may be transferred to the set of aggregate vials 800 via the one or more aggregate syringes 2700, thereby defining the set of aliquots of the aggregate 17 derived from the whole blood 11. More specifically, a needle 2711 connected to an end connector 2710 of each of the one or more aggregate syringes 2700 may be inserted through the extraction port 2670 of the aggregate reservoir 2600. With the needle 2711 of each of the one or more aggregate syringes 2700 so inserted, a plunger 2770 thereof may then be operated to retrieve aggregate 17 from within the aggregate reservoir 2600, and to subsequently transfer the aggregate 17 to the set of aggregate vials 800.

Again, as additionally depicted, each of the aggregate vials 800 may carry an ID tag 5800 that is indicative of the identity of the donor 10. In a manner similar to the ID tag 5600, it may be that the ID tags 5800, regardless of the technology on which they are based, may be printed and/or caused to store an identifier at a time on or about when the whole blood 11 is drawn from the donor 10.

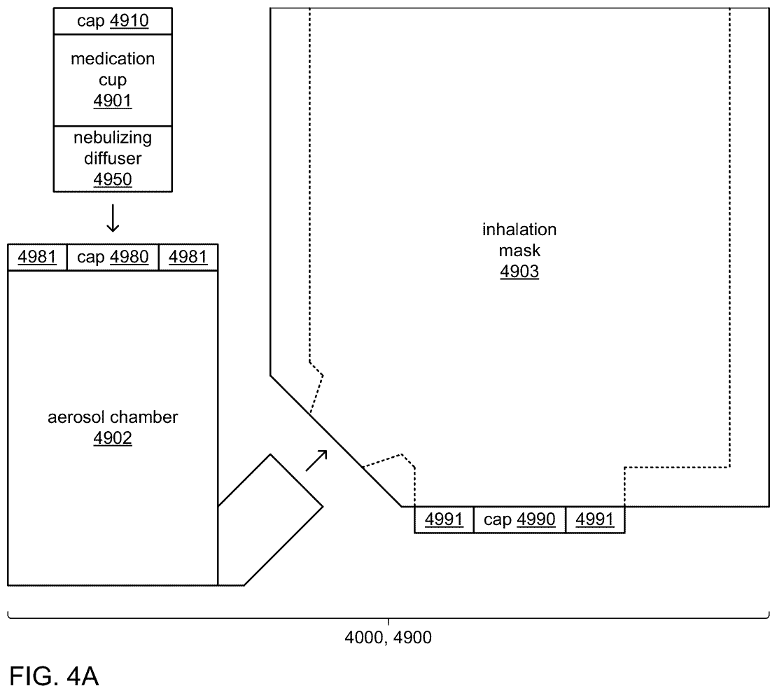

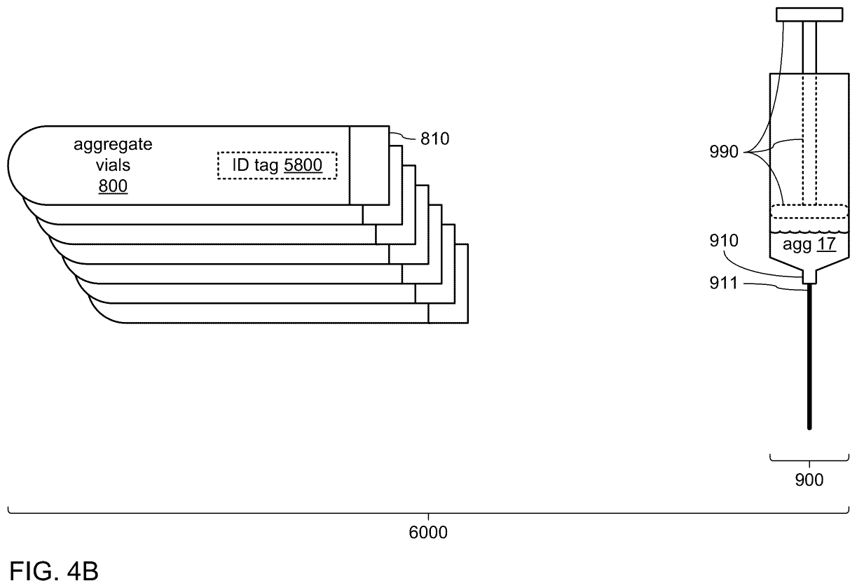

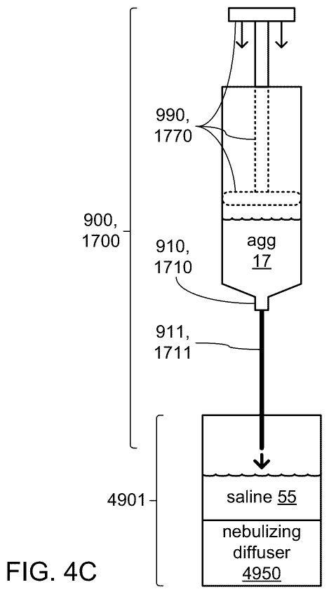

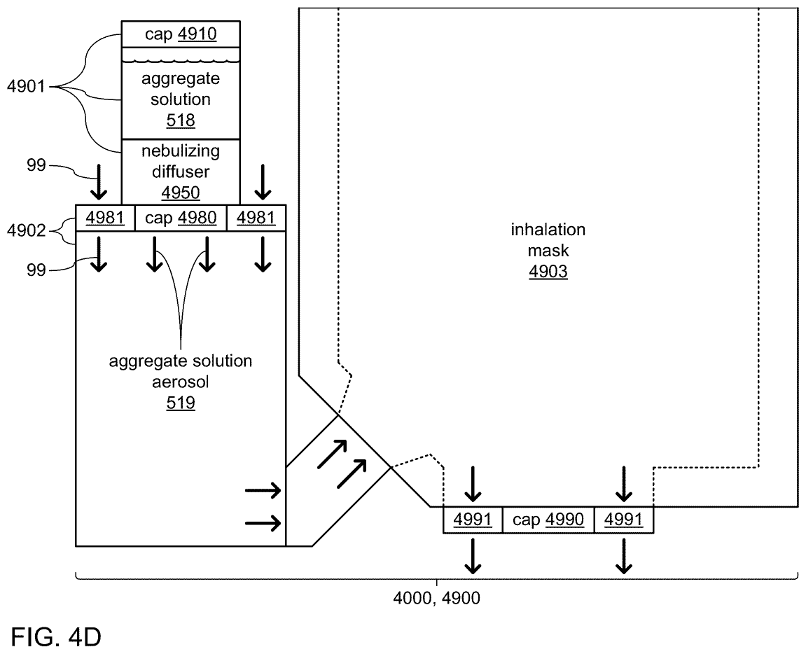

FIGS. 4A-D, taken together, present aspects of the inhalation system 4000 in greater detail. More specifically, FIG. 4A introduces an embodiment of a nebulizer 4900 of the inhalation system 4000; FIGS. 4B-C depict aspects of the provision of the aggregate 17 of α2M molecules 15 to the nebulizer 4900; and FIG. 4D depicts aspects of the use of the nebulizer 4900 to administer the aggregate 17 to the patient 90.

Turning to FIG. 4A, as depicted, the nebulizer 4900 of the inhalation system 4000 may include a combination of a medication cup 4901, an aerosol chamber 4902 and an inhalation mask 4903. Both of the medication cup 4901 and the aerosol chamber 4902 may be of a generally elongate cylindrical shape such that each defines a pair of ends. It should be noted that the particular example of the nebulizer 4900 that is depicted may be physically shaped and/or sized for use with such creatures as a camel or horse. More specifically, the inhalation mask 4903 may be physically configured to fit over the muzzle or snout of the patient 90 where the patient 90 is such a creature to thereby intercept the flow of inhalation of air by the patient 90 as part of adding a flow of aerosol thereto. Still more specifically, in some embodiments, the nebulizer 4900 may be one of various models offered by FLEXINEB® of Union City, Tennessee, USA. However, other embodiments are possible in which the nebulizer 4900 may be physically shaped and/or sized for use with other creatures having mouths, muzzles, noses, snouts, etc. that may differ considerably from those of a camel or horse.

One end of the medication cup 4901 may be closeable with a cap 4910. The other end of the medication cup 4901 may incorporate a nebulizing diffuser 4950. In various embodiments, the nebulizing diffuser 4950 may employ any of a variety of techniques to generate an aerosol of whatever liquid may be placed within the interior volume of the medication cup 4901 between the cap 4910 and the nebulizing diffuser 4950. Such techniques for aerosolizing such liquids, include, but are not limited to, heating, ultrasonic vibration, etc.

One end of the aerosol chamber 4902 may be closeable with a cap 4980 that may be configured to enable the connection of the end (or other portion) of the medication cup 4901 that incorporates the nebulizing diffuser 4950 thereto in a manner that allows aerosols generated by the nebulizing diffuser 4950 to enter the interior volume of the aerosol chamber 4902. The cap 4980 may additionally include one or more inhalation inlets 4981 that may incorporate one-way valves that enable the entry of surrounding air into the aerosol chamber 4902, while preventing (or at least restricting) the release of the aerosols generated by the nebulizing diffuser 4950 into the surrounding air. The other end of the aerosol chamber 4902 may be configured to be connected to the inhalation mask 4903 in a manner that allows relatively free passage of air and aerosols from within the aerosol chamber 4902 and into the inhalation mask 4903.

The inhalation mask 4903 may incorporate an opening configured to allow the muzzle or snout of such a creature as a camel or a horse (i.e., where the patient 90 is such a creature) to enter into the interior space thereof, and may be further configured for connection to the aerosol chamber 4902 to receive air and aerosols therefrom. There may be still another opening formed in the inhalation mask 4903 that may be closeable with a cap 4990 that may additionally include one or more exhalation outlets 4991 that may incorporate one-way valves that enable the release of gases exhaled from the nose and/or mouth of the muzzle or snout of the patient 90 into the surrounding air, while preventing (or at least restricting) the entry of the surrounding air directly into the inhalation mask 4903. In this way, when the patient 90 inhales while wearing the inhalation mask 4903, the inhaled air is forced to pass through the aerosol chamber 4902, thereby enabling an aerosol generated by the nebulizing diffuser 4950 to be drawn into the inhalation mask 4903 along with the inhaled air.

Turning to FIG. 4B, following the generation of the aggregate 17 through the aggregation of α2M molecules 15 from the whole blood 11 of a donor 10, as described in reference to FIGS. 2A-K or as described in reference to FIGS. 3A-D, a portion of such aggregate 17 may be prepared for being transferred to the nebulizer 4900.

More specifically, and referring back to either of FIG. 2K or FIG. 3D, in addition to FIG. 4B, it may be that a portion of the aggregate 17 (e.g., an aliquot from a single aggregate vial 800) is to be administered to the patient 90 by inhalation immediately (e.g., during the same visit in which the aggregation of the α2M molecules 15 to generate the aggregate 17 is performed). In such a situation, it may be that the very same aggregate syringe 1700 (or one of the very same aggregate syringes 1700) that is used to transfer the aggregate 17 into the set of vials 800 is also used to transfer that portion of the aggregate 17 to the nebulizer 4900.

Alternatively, and as also depicted in FIG. 4B, it may be that at least some of the aggregate 17 that is stored within at least one of the vials 800 is to be administered to the patient 90 by inhalation. In such a situation, it may be that a different aggregate syringe 900 is used to transfer such an amount of the aggregate 17 from one or more of the aggregate vials 800, and into the nebulizer 4900.

It is envisioned that the amount of the aggregate 17 that is generated from the whole blood 11 will be more than enough to support multiple instances of administering the aggregate 17 to the patient 90 via inhalation. Thus, it is envisioned that at least one aggregate vial 800 will be filled with an aliquot of the aggregate 17 that is not meant to be used immediately in an administration of the aggregate 17 to the patient 90.

Experiments conducted so far by the inventors in treating horses for EA have shown considerable effectiveness with 6 doses delivered at a rate of 1 dose every other day. As will shortly be explained, in these experiments, each dose is diluted with an equal volume of either 0.9% saline solution or lactated Ringer's solution. In these studies, all other medications and/or treatments for EA (if any) were discontinued for all horses. The results were promising, with most of the horses showing improvement. One horse experienced a transient episode of increased coughing associated with one instance of nebulization, and no horses demonstrated epistaxis. Subsequently, many of the horses demonstrated a decrease in clinical signs of EA for a period of 1 to 6 months, and while not being provided with any other medications. Several of the horses that began the study with more severe cases of EA have been able to be maintained with a single nebulization once every 4 weeks. A small number of these horses have additionally been provided with intermittent doses of medications to treat specific clinical signs associated with EA.

It should be noted that further experiments are planned by Applicant to attempt to derive more optimal parameters for the delivery of treatment, including and not limited to, the quantity of aggregate 17 to be delivered overall and/or for each aliquot, the number of doses (aliquots) to be delivered and/or their frequency, the ratio of mixture with saline solution and/or another form of dilution liquid, etc.

Turning to FIG. 4C, as part of preparing to use the nebulizer 4900 to deliver a dose of the aggregate 17, the cap 4910 may be removed from the medication cup 4901 to provide access to the interior volume therein, and an amount of saline solution 55 may be deposited therein. Also, an amount of the aggregate 17 (e.g., some or all of an aliquot contained within one of the aggregate vials 800) may also be deposited within the interior volume of the medication cup 4901. As previously discussed, successful results in treating at least EA have been achieved through use of an equal parts mixture of aggregate 17 and 0.9% saline solution. However, as also previously discussed, further planned experimentation may demonstrate that a different mixture ratio and/or the use of a different solution begets still better results.

Turning to FIG. 4D, after the aggregate 17 and the saline solution 55 have been deposited within the medication cup 4901, thereby creating an α2M solution 518, the cap 4910 may be reinstalled thereon, and the medication cup 4901 may be coupled to the cap 4980 of the aerosol chamber 4902. With the aerosol chamber 4902 also connected to the inhalation mask 4903, and with the inhalation mask 4903 being worn by the patient 90, the nebulizing diffuser 4950 may then be operated to begin generating an α2M solution aerosol 519 from the α2M solution 518. As the α2M solution aerosol 519 is so generated, it is released into the aerosol chamber 4902, thereby building up an amount thereof that is retained within the aerosol chamber 4902 until the patient 90 takes a breath. This results in much of the α2M solution aerosol 519 being drawn, along with surrounding air 99 that enters into the aerosol chamber 4902 through inhalation inlets 4981, into the inhalation mask 4903, where the patient 90 inhales both.

As the patient 90 exhales, the valves within the inhalation inlets 4981 and/or within the exhalation outlets 4991 cooperate with the pressure behind the exhalation by the patient 90 to stop the flow of air 99 and α2M solution aerosol 519 into the inhalation mask 4903 from the aerosol chamber 4902, thereby allowing another amount of the α2M solution aerosol 519 to collect within the aerosol chamber 4902 in preparation for the next inhalation by the patient 90. The valves within the inhalation inlets 4981 and/or the exhalation outlets 4991 also cooperate with the pressure behind the exhalation by the patient 90 to cause the exhaled gases from the patient 90 to be released from within the inhalation mask 4903 through the exhalation outlets 4991.

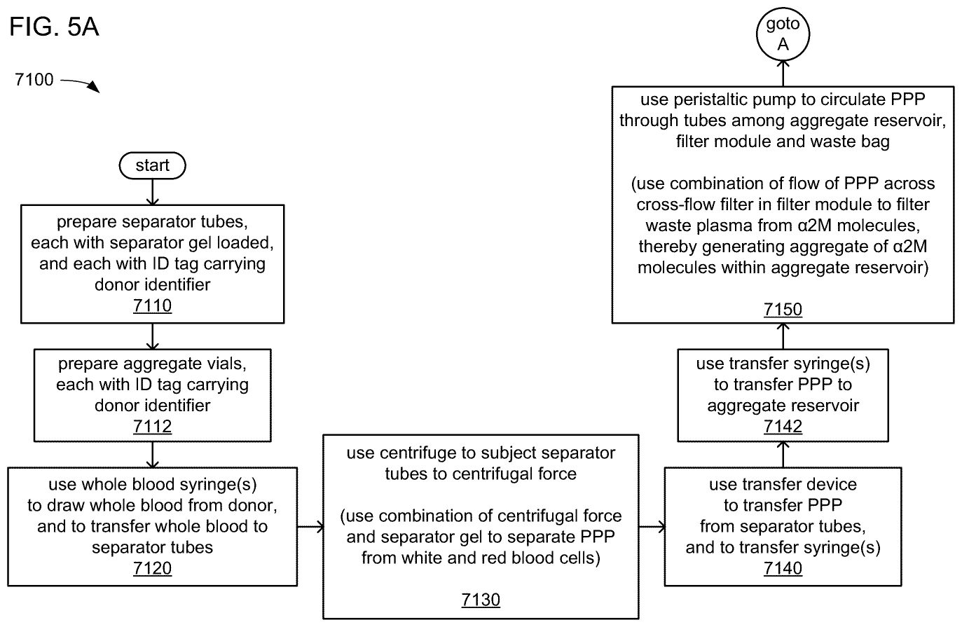

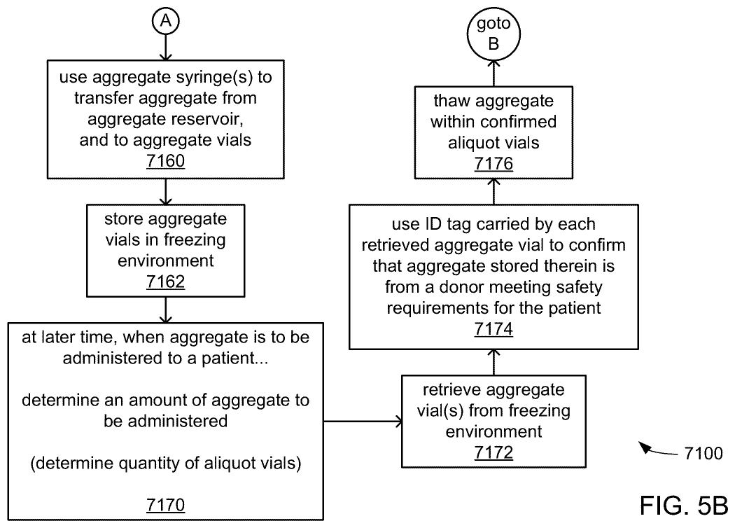

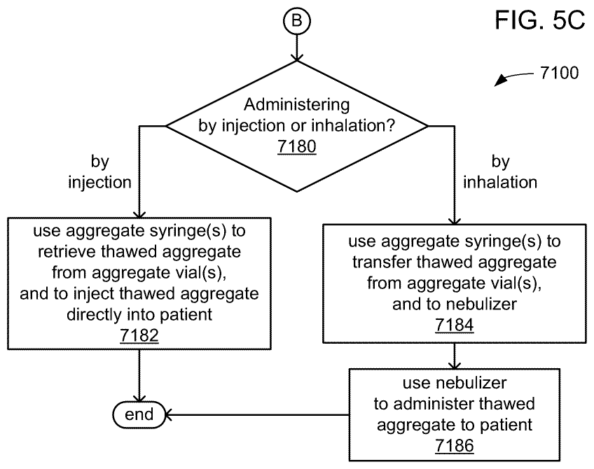

FIGS. 5A-C, taken together, present a flowchart 7100 depicting aspects of the operation of an example of the system 6000 of FIG. 1D (and also of FIGS. 3A-D and 4A-D) for aggregating and administering α2M molecules 15 in an allogeneic or xenogeneic manner via either injection or inhalation.

At 7110, a quantity of separator tubes may be prepared with separator gel deposited within each, and with each carrying an ID tag that carries an identifier of a donor (e.g., the separator tubes 1200, each with separator gel 1250 therein, and each carrying an ID tag 5200 with an identifier of one of multiple donors 10). At 7112, a quantity of aggregate vials may be prepared with each carrying an ID tag that carries the identifier of the donor (e.g., the aggregate vials 800, each carrying an ID tag 5800).

At 7120, one or more whole blood syringes may be used to draw whole blood from the donor (e.g., the one or more whole blood syringes 100 used to draw whole blood 11 from the one of the multiple donors 10), and to transfer that whole blood to the separator tubes.

At 7130, the separator tubes may be placed within a centrifuge (e.g., the centrifuge 1500), and the centrifuge may be operated to exert centrifugal force on the separator tubes. In this way, a combination of the exerted centrifugal force and the separator gel within each separator tube may be used to separate platelet poor plasma (PPP) containing α2M molecules from other components of the whole blood (e.g., separating the PPP 13 from the red and white blood cells 12).

At 7140, a transfer device may be used to transfer the PPP from within each of the separator tubes to one or more transfer syringes (e.g., one of the transfer devices 1300a, 1300b or 1300c, to one or more of the transfer syringes 1400). At 7242, the one or more transfer syringes may be used to transfer the plasma into an aggregate reservoir (e.g., the α2M reservoir 2600).

At 7150, the aggregate reservoir may be connected to a filtration module incorporating a cross-flow filter and a waste bag (e.g., the filtration module 2300 and the waste bag 2400), and a peristaltic pump may then be used to circulate the plasma among the aggregate reservoir, the filter module and the waste bag (e.g., the peristaltic pump 2500). In this way, the PPP is repeatedly circulated across a face of filter media of the cross-flow filter of the filter module for a period of time sufficient to cause α2M molecules to remain within the aggregate reservoir, while other components of the PPP permeate through the cross-flow filter of the filter module, and into the waste bag. In this way, an aggregate of the α2M molecules (e.g., the aggregate 17) is generated from the PPP within the aggregate reservoir.

At 7160, one or more aggregate syringes may be used to transfer the aggregate of the α2M molecules from the aggregate reservoir, and to the aggregate vials (e.g., the aggregate syringes 1700 transferring aggregate 17 to the aggregate vials 800). With the aggregate so transferred to the aggregate vials, at 7162, the aggregate vials may be stored within a freezing environment (e.g., a freezer) to preserve the aggregate in storage for an extended period of time, or until needed to treat a patient.

Thus, at 7170, at a later time, an amount of aggregate of α2M molecules to administer to a patient in that dose may be determined. At 7172, a quantity of aggregate vials required to provide that dose to a patient may be retrieved from the freezing environment in which the aggregate vials were stored.

At 7174, the ID tag carried by each retrieved aggregate vial may be used to confirm that the aggregate stored therein is from a donor meeting the safety requirements for the patient. At 7176, each of such retrieved and confirmed aggregate vials may be thawed as part of preparing the aggregate stored therein for being administered to the patient.

If, at 7180, the thawed aggregate is to be administered by injection, then at 7182, one or more aggregate syringes (e.g., the aggregate syringes 900) may be used to so inject the patient with the thawed aggregate of α2M molecules.

However, if, at 7180, the thawed aggregate is to be administered by inhalation, then at 7184, one or more aggregate syringes may be used to transfer the thawed aggregate to a nebulizer (e.g., the nebulizer 4900). At 7186, the nebulizer may be used to administer the thawed aggregate of α2M molecules to the patient via inhalation.

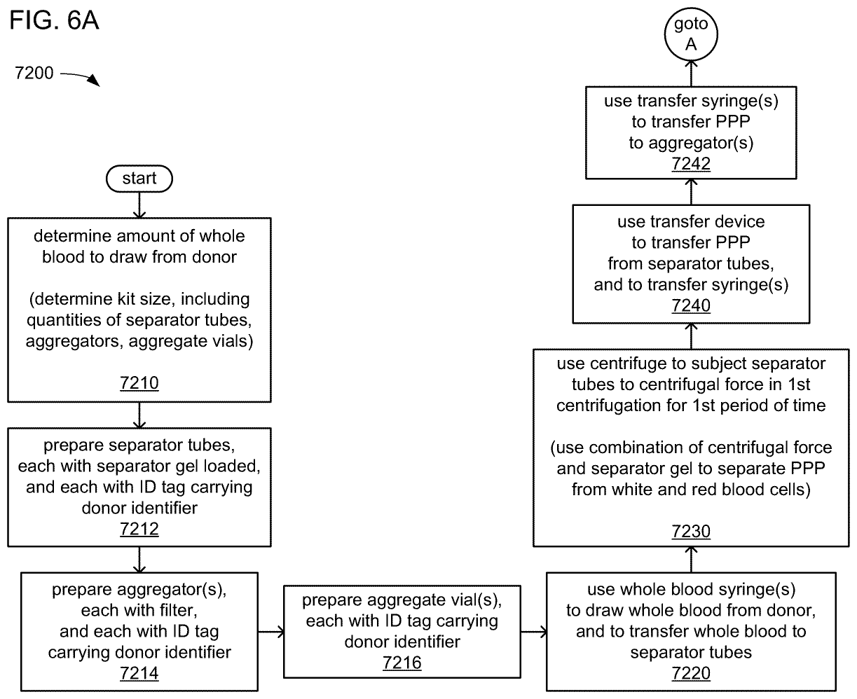

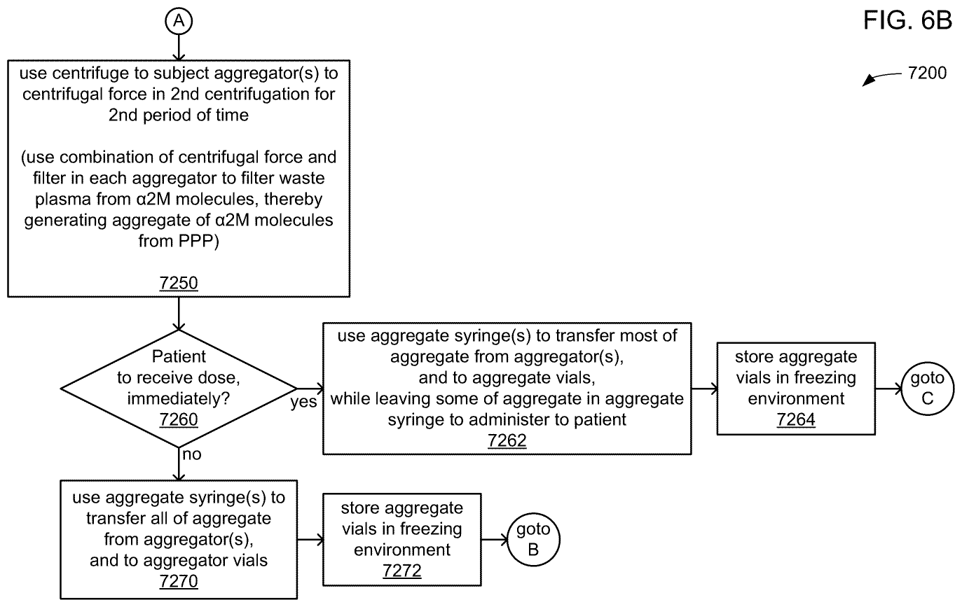

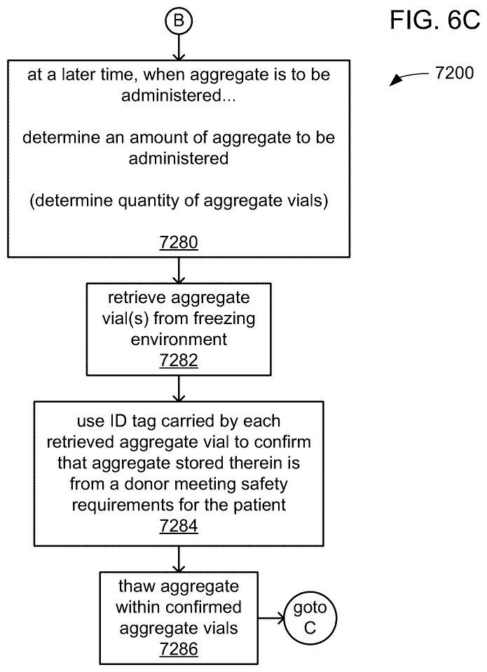

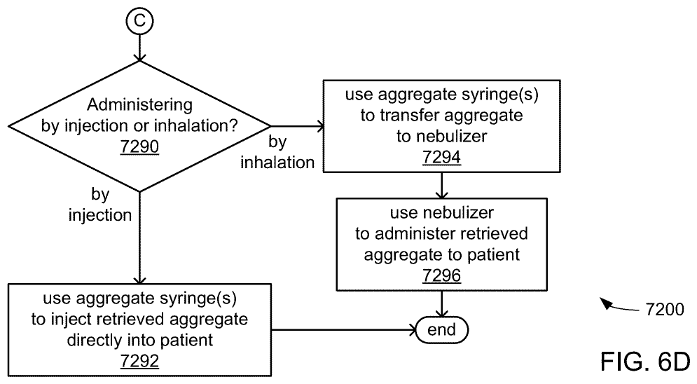

FIGS. 6A-D, taken together, present a flowchart 7200 depicting aspects of the operation of an example of the system 6000 of FIG. 1B (and also of FIGS. 2A-K and 4A-D) for aggregating and administering α2M molecules 15 in an allogeneic or xenogeneic manner via either injection or inhalation.

At 7210, the amount of whole blood required to treat a respiratory condition of a patient (e.g., the patient 90—e.g., a camel, a dog, a horse, a human, a pig, etc.) may be determined. More precisely, a kit may be selected that includes sufficient quantities of separator tubes, aggregators and aggregate vials to support the provision of enough aggregate of α2M molecules to treat the patient from the whole blood of a donor (e.g., the aggregate 17 of α2M molecules 15 from the whole blood 11 of one of the multiple donors 10).

At 7212, the separator tubes may be prepared with separator gel deposited within each, and with each carrying an ID tag that carries an identifier of the patient (e.g., the separator tubes 1200, each with separator gel 1250 therein, and each carrying an ID tag 5200). At 7214, each aggregator of the one or more aggregators may be prepared with a filter carried within each, and with each carrying an ID tag that carries the identifier of the patient (e.g., the one or more aggregators 1600, each with a filter 1650 therein, and each carrying an ID tag 5600). At 7216, the aggregate vials may be prepared with each carrying an ID tag that carries the identifier of the patient (e.g., the aggregate vials 800, each carrying an ID tag 5800).

At 7220, one or more whole blood syringes may be used to draw whole blood from the donor (e.g., the one or more whole blood syringes 100), and to transfer that whole blood to the separator tubes.

At 7230, the separator tubes may be placed within a centrifuge (e.g., the centrifuge 1500), and the centrifuge may be operated to exert centrifugal force on the separator tubes in a first stage of centrifuging (i.e., a first centrifugation) for a first period of time. In this way, a combination of the exerted centrifugal force and the separator gel within each separator tube may be used to separate platelet poor plasma (PPP) containing α2M molecules from other components of the whole blood (e.g., separating the PPP 13 from the red and white blood cells 12).

At 7240, a transfer device may be used to transfer the PPP from within each of the separator tubes to one or more transfer syringes (e.g., one of the transfer devices 1300a, 1300b or 1300c, to one or more of the transfer syringes 1400). At 7242, the one or more transfer syringes may be used to transfer the PPP into one or more aggregators.

At 7250, the one or more aggregators may be placed within the centrifuge, and the centrifuge may be operated to exert centrifugal force on the aggregator(s) in a second stage of centrifuging (i.e., a second centrifugation) for a second period of time. In this way, a combination of the exerted centrifugal force and the filter within each aggregator that is filled with PPP may be used to filter waste plasma from the α2M molecules, thereby generating an aggregate of α2M molecules from the PPP (e.g., generating the aggregate 17). Again, where just one aggregator is filled with PPP, a counterbalancing weight, or other aggregator that is filled with water or another substance to serve as a counterbalancing weight, may be required to balance the centrifuge.

If, at 7260, the patient is to receive a dose of the aggregate, immediately, then at 7262, one or more aggregate syringes may be used to transfer most of the aggregate from the aggregator(s), and to the aggregate vials (e.g., the aggregate syringes 1700 transferring aggregate 17 to the aggregate vials 800). However, some remaining amount of the aggregate may be retained within an aggregate syringe for use in providing immediate treatment to the patient. With the aggregate so transferred to the aggregate vials, at 7264, the aggregate vials may be stored within a freezing environment (e.g., a freezer) to preserve the aggregate therein in storage for an extended period of time. With the most the aggregate so frozen, the immediate administration of aggregate to the patient may be performed, starting at 7290.

However, if, at 7260, the patient is not to receive a dose of the aggregate, immediately, then at 7270, one or more aggregate syringes may be used to transfer all of the aggregate from the aggregator(s), and to the aggregate vials. With the aggregate so transferred, at 7272, the aggregate vials may be stored within a freezing environment. With the aggregate so frozen, preparations for an administration of aggregate to the patient may begin at a later time, starting at 7280.

Thus, at 7280, at a later time, an amount aggregate to administer to the patient in that dose may be determined. At 7282, a quantity of aggregate vials required to provide the dose to the patient may be retrieved from the freezing environment in which the aggregate vials were stored.

At 7284, the ID tag carried by each retrieved aggregate vial may be used to confirm that the aggregate stored therein is from a donor meeting the safety requirements for the patient. At 7286, each of such retrieved and confirmed aggregate vials may be thawed as part of preparing the aggregate stored therein for being administered to the patient.

If, at 7290, the thawed aggregate is to be administered by injection, then at 7292, one or more aggregate syringes (e.g., the aggregate syringes 900) may be used to so inject the patient with the thawed aggregate.

However, if, at 7290, the thawed aggregate is to be administered by inhalation, then at 7294, one or more aggregate syringes may be used to transfer the thawed aggregate to a nebulizer (e.g., the nebulizer 4900). At 7296, the nebulizer may be used to administer the thawed aggregate to the patient via inhalation.

There is thus disclosed a system and a method for aggregating α2M molecules from whole blood, and then administering those α2M molecules to a patient to treat either a musculoskeletal or respiratory condition.

A method for treating a medical condition of a patient with at least Alpha-2 Macroglobulin (α2M) molecules in an allogeneic or xenogeneic manner includes: drawing whole blood from a donor; separating platelet poor plasma (PPP) containing α2M molecules from other components of the whole blood; filtering waste plasma from the PPP to generate an aggregate of the α2M molecules; and administering at least some of the aggregate to the patient via injection or inhalation, wherein the donor and the patient are different individuals.

The medical condition may include at least one of a musculoskeletal condition entailing damage to musculoskeletal tissue of the patient or a respiratory condition entail damage to tissue of a respiratory tract of the patient.

The donor and the patient may be of the same species.

The donor and the patient may be of different species.

Drawing the whole blood from the donor may include: using a whole blood syringe including a hollow needle to draw the whole blood from the donor; and partially pre-filling the whole blood syringe with an anticoagulant before using the whole blood syringe to draw the whole blood from the donor.

Separating the PPP from other components of the whole blood may include: depositing the whole blood into 1 separator tube, wherein each separator tube of the at least one separator tube contains an amount of separator gel; and subjecting the at least one separator tube to a first centrifugal force in a first centrifuging stage for a first predetermined period of time to cause a combination of the first centrifugal force and the separator gel within each separator tube of the at least one separator tube to separate the PPP from red blood cells and white blood cells of the whole blood within the at least one separator tube.

Filtering the waste plasma from the PPP may include: following the first centrifuging stage, transferring the PPP from the at least one separator tube and into at least one aggregator, wherein each aggregator of the at least one aggregator includes a filter; and subjecting the at least one aggregator to a second centrifugal force in a second centrifuging stage for a second predetermined period of time to cause a combination of the second centrifugal force and the filter within each aggregator of the at least one aggregator to filter the waste plasma from the PPP to generate the aggregate within the at least one aggregator.

Transferring the PPP from the at least one separator tube and into the at least one aggregator may include: coupling a transfer syringe to a syringe port of a transfer device, wherein the syringe port is configured to receive an end connector of the transfer syringe that is configured to be coupled to a transfer needle; coupling each separator tube of the at least one separator tube, one at a time, to a separator tube port of the transfer device, wherein the separator tube port includes at least one hollow needle configured to penetrate the cap of each separator tube to couple the separator tube to the syringe port of the transfer device; while each separator tube of the at least one separator tube is coupled to the separator tube port, operating a plunger of the transfer syringe to withdraw at least some of the PPP from within the separator tube and into the transfer syringe through the transfer device; and following the transfer of PPP from each separator tube of the at least one separator tube, using the transfer syringe, with the transfer needle coupled to the end connector, to inject the PPP within the transfer syringe into the at least one aggregator.

The filter of each aggregator of the at least one aggregator may have a molecular weight cut off ranging from 100 kD to 500 kD.

Filtering waste plasma from the PPP may include: following the first centrifuging stage, transferring the PPP from the at least one separator tube and into an aggregate reservoir; and using a peristaltic pump to circulate the PPP among the aggregate reservoir, a cross-flow filter and a waste bag to cause the waste plasma to pass through the cross-flow filter and into the waste bag, while the α2M molecules remain within the aggregate reservoir.

The cross-flow filter may be selected to prevent molecules of greater than 500 kDa from passing therethrough.

Administering at least some of the aggregate to the patient via inhalation may include administering at least some of the aggregate to the patient using a nebulizer.

The method may further include storing a remainder of the aggregate within at least one aggregate vial in a freezing environment to preserve the remainder of the aggregate for use in another administration of the aggregate to the patient at a later time.

The aggregate may include at least one of serum albumin molecules or serotransferrin molecules from the whole blood along with the α2M molecules.

A kit for treating a respiratory condition of a patient with at least Alpha-2 Macroglobulin (α2M) molecules in an allogeneic or xenogeneic manner includes at least one separator tube, wherein each separator tube of the at least one separator tube includes: an elongate transparent tube that defines an opening at one end that is sealed with a cap that is penetrable to receive whole blood drawn from a donor; and an amount of separator gel disposed within the separator tube to cooperate with a first centrifugal force exerted on the separator tube for a first period of time during a first centrifuging stage to separate platelet poor plasma (PPP) containing α2M molecules from other components of the whole blood. The kit also includes at least one aggregator, wherein each aggregator of the at least one aggregator includes: a filter; a first cylinder defined by a first cylindrical wall having a first end that is configured to be closable with a septum cap that is penetrable to receive the PPP following the first centrifuging stage, and having a second end that is closed with the filter; and a second cylinder defined by a second cylindrical wall having a first end that is closed where the second cylindrical wall narrows to form a conically-shaped end portion, and having a second end that defines an opening that is configured to be coupled to the filter in a manner that causes a first interior volume of the first cylinder and a second interior volume of the second cylinder to be separated by the filter, wherein the filter is configured to cooperate with a second centrifugal force exerted on the aggregator for a second period of time during a second centrifuging stage to filter waste plasma from the PPP to generate an aggregate of α2M molecules. The kit also includes a transfer device, including: a separator tube port configured to receive each separator tube of the at least one separator tube, one at a time, wherein the separator tube port includes at least one hollow needle configured to penetrate the cap of each separator tube to couple the separator tube to a syringe port of the transfer device; and a syringe port configured to receive an end connector of a transfer syringe that is configured to be coupled to a transfer needle, wherein, following the first centrifuging stage and prior to the second centrifuging stage; while each separator tube of the at least one separator tube is coupled to the separator tube port, a plunger of the transfer syringe is operable to withdraw at least some of the PPP from within the separator tube and into the transfer syringe through the transfer device; and following the transfer of PPP from each separator tube of the at least one separator tube, and with the transfer needle coupled to the end connector to penetrate the septum cap of each aggregator of the at least one aggregator, the plunger of the transfer syringe is operable to inject the PPP within the transfer syringe into the at least one aggregator.

Each aggregator of the at least one aggregator may be configured to: receive the injection of the PPP within the first interior volume within the first cylinder; and filter the waste plasma from the PPP to generate the aggregate within the first interior volume, while leaving the waste plasma within the second interior volume.

The septum cap may further include a third cylindrical wall configured to serve as an extension to the first cylindrical wall to increase a volume of the first interior volume when the first end of the first cylindrical wall is closed with the septum cap.

The filter of each aggregator of the at least one aggregator may have a molecular weight cut off ranging from 100 kD to 500 kD.

The kit may further include a nebulizer configured to be provided with the aggregate, and to administer the aggregate to the patient via inhalation.

The aggregate may include at least one of serum albumin molecules or serotransferrin molecules from the whole blood along with the α2M molecules.

Various other components may be included and called upon for providing for aspects of the teachings herein. For example, additional materials, combinations of materials, and/or omission of materials may be used to provide for added embodiments that are within the scope of the teachings herein.

Standards for performance, selection of materials, functionality, and other discretionary aspects are to be determined by a user, designer, manufacturer, or other similarly interested party. Any standards expressed herein are merely illustrative and are not limiting of the teachings herein.

When introducing elements of the present disclosure or the embodiment(s) thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

While the disclosure has been described with reference to illustrative embodiments, it will be understood by those skilled in the art that various changes may be made and equivalents may be substituted for elements thereof without departing from the scope of the invention. In addition, many modifications will be appreciated by those skilled in the art to adapt a particular instrument, situation or material to the teachings of the invention without departing from the essential scope thereof. Therefore, it is intended that the claimed invention not be limited to the particular embodiment disclosed as the best mode contemplated for carrying out this invention, but that the invention will include all embodiments falling within the scope of the appended claims.

1. A method for treating a medical condition of a patient with at least Alpha-2 Macroglobulin (α2M) molecules in an allogeneic or xenogeneic manner, the method comprising:
drawing whole blood from a donor;
separating platelet poor plasma (PPP) containing α2M molecules from other components of the whole blood;
filtering waste plasma from the PPP to generate an aggregate of the α2M molecules; and
administering at least some of the aggregate to the patient via injection or inhalation, wherein the donor and the patient are different individuals.

2. The method of claim 1, wherein the medical condition comprises at least one of:
a musculoskeletal condition entailing damage to musculoskeletal tissue of the patient; or
a respiratory condition entail damage to tissue of a respiratory tract of the patient.

3. The method of claim 1, wherein the donor and the patient are of the same species.

4. The method of claim 1, wherein the donor and the patient are of different species.

5. The method of claim 1, wherein drawing the whole blood from the donor comprises:
using a whole blood syringe comprising a hollow needle to draw the whole blood from the donor; and
partially pre-filling the whole blood syringe with an anticoagulant before using the whole blood syringe to draw the whole blood from the donor.

6. The method of claim 1, wherein separating the PPP from other components of the whole blood comprises:
depositing the whole blood into 1 separator tube, wherein each separator tube of the at least one separator tube contains an amount of separator gel; and
subjecting the at least one separator tube to a first centrifugal force in a first centrifuging stage for a first predetermined period of time to cause a combination of the first centrifugal force and the separator gel within each separator tube of the at least one separator tube to separate the PPP from red blood cells and white blood cells of the whole blood within the at least one separator tube.

7. The method of claim 6, wherein filtering the waste plasma from the PPP comprises:
following the first centrifuging stage, transferring the PPP from the at least one separator tube and into at least one aggregator, wherein each aggregator of the at least one aggregator comprises a filter; and
subjecting the at least one aggregator to a second centrifugal force in a second centrifuging stage for a second predetermined period of time to cause a combination of the second centrifugal force and the filter within each aggregator of the at least one aggregator to filter the waste plasma from the PPP to generate the aggregate within the at least one aggregator.

8. The method of claim 7, wherein transferring the PPP from the at least one separator tube and into the at least one aggregator comprises:
coupling a transfer syringe to a syringe port of a transfer device, wherein the syringe port is configured to receive an end connector of the transfer syringe that is configured to be coupled to a transfer needle;
coupling each separator tube of the at least one separator tube, one at a time, to a separator tube port of the transfer device, wherein the separator tube port comprises at least one hollow needle configured to penetrate the cap of each separator tube to couple the separator tube to the syringe port of the transfer device;
while each separator tube of the at least one separator tube is coupled to the separator tube port, operating a plunger of the transfer syringe to withdraw at least some of the PPP from within the separator tube and into the transfer syringe through the transfer device; and
following the transfer of PPP from each separator tube of the at least one separator tube, using the transfer syringe, with the transfer needle coupled to the end connector, to inject the PPP within the transfer syringe into the at least one aggregator.

9. The method of claim 7, wherein the filter of each aggregator of the at least one aggregator has a molecular weight cut off ranging from 100 kD to 500 kD.

10. The method of claim 1, wherein filtering waste plasma from the PPP comprises:
following the first centrifuging stage, transferring the PPP from the at least one separator tube and into an aggregate reservoir; and
using a peristaltic pump to circulate the PPP among the aggregate reservoir, a cross-flow filter and a waste bag to cause the waste plasma to pass through the cross-flow filter and into the waste bag, while the α2M molecules remain within the aggregate reservoir.

11. The method of claim 10, wherein the cross-flow filter is selected to prevent molecules of greater than 500 kDa from passing therethrough.

12. The method of claim 1, wherein administering at least some of the aggregate to the patient via inhalation comprises administering at least some of the aggregate to the patient using a nebulizer.

13. The method of claim 1, further comprising storing a remainder of the aggregate within at least one aggregate vial in a freezing environment to preserve the remainder of the aggregate for use in another administration of the aggregate to the patient at a later time.

14. The method of claim 13, wherein the aggregate comprises at least one of serum albumin molecules or serotransferrin molecules from the whole blood along with the α2M molecules.

15. A kit for treating a respiratory condition of a patient with at least Alpha-2 Macroglobulin (α2M) molecules in an allogeneic or xenogeneic manner, the kit comprising:
at least one separator tube, wherein each separator tube of the at least one separator tube comprises:
an elongate transparent tube that defines an opening at one end that is sealed with a cap that is penetrable to receive whole blood drawn from a donor; and
an amount of separator gel disposed within the separator tube to cooperate with a first centrifugal force exerted on the separator tube for a first period of time during a first centrifuging stage to separate platelet poor plasma (PPP) containing α2M molecules from other components of the whole blood;
at least one aggregator, wherein each aggregator of the at least one aggregator comprises:
a filter;
a first cylinder defined by a first cylindrical wall having a first end that is configured to be closable with a septum cap that is penetrable to receive the PPP following the first centrifuging stage, and having a second end that is closed with the filter; and
a second cylinder defined by a second cylindrical wall having a first end that is closed where the second cylindrical wall narrows to form a conically-shaped end portion, and having a second end that defines an opening that is configured to be coupled to the filter in a manner that causes a first interior volume of the first cylinder and a second interior volume of the second cylinder to be separated by the filter, wherein the filter is configured to cooperate with a second centrifugal force exerted on the aggregator for a second period of time during a second centrifuging stage to filter waste plasma from the PPP to generate an aggregate of α2M molecules; and
a transfer device, comprising:
a separator tube port configured to receive each separator tube of the at least one separator tube, one at a time, wherein the separator tube port comprises at least one hollow needle configured to penetrate the cap of each separator tube to couple the separator tube to a syringe port of the transfer device; and
a syringe port configured to receive an end connector of a transfer syringe that is configured to be coupled to a transfer needle, wherein, following the first centrifuging stage and prior to the second centrifuging stage:
while each separator tube of the at least one separator tube is coupled to the separator tube port, a plunger of the transfer syringe is operable to withdraw at least some of the PPP from within the separator tube and into the transfer syringe through the transfer device; and
following the transfer of PPP from each separator tube of the at least one separator tube, and with the transfer needle coupled to the end connector to penetrate the septum cap of each aggregator of the at least one aggregator, the plunger of the transfer syringe is operable to inject the PPP within the transfer syringe into the at least one aggregator.

16. The kit of claim 15, wherein each aggregator of the at least one aggregator is configured to:
receive the injection of the PPP within the first interior volume within the first cylinder; and
filter the waste plasma from the PPP to generate the aggregate within the first interior volume, while leaving the waste plasma within the second interior volume.

17. The kit of claim 15, wherein the septum cap further comprises a third cylindrical wall configured to serve as an extension to the first cylindrical wall to increase a volume of the first interior volume when the first end of the first cylindrical wall is closed with the septum cap.

18. The kit of claim 15, wherein the filter of each aggregator of the at least one aggregator has a molecular weight cut off ranging from 100 kD to 500 kD.

19. The kit of claim 15, further comprising a nebulizer configured to be provided with the aggregate, and to administer the aggregate to the patient via inhalation.

20. The kit of claim 15, wherein the aggregate comprises at least one of serum albumin molecules or serotransferrin molecules from the whole blood along with the α2M molecules.
